(19) United States
(12) Patent Application Publication
(10) Pub. No.: US 2008/0025981 A1
(43) Pub. Date: Jan. 31, 2008

(54) Cancer-linked genes as targets for chemotherapy

(76) Inventors: Paul E. Young, Sudbury, MA (US); Reinhard Ebner, Gaithersburg, MD (US); Zoe Weaver, Ijamsville, MD (US); Jeffrey W. Strovel, Laurel, MD (US); Stephen K. Horrigan, Poolesville, MD (US); Martin Shea, Gaithersburg, MD (US); Bernd Weigle, Hanau (DE); Michael Rieger, Dresden (DE); Jennifer A. Rick, Germantown, MD (US); Colyn B. Cain, Bethesda, MD (US)

Correspondence Address: Alan J. Grant, Esq.;c/o Carella, Byrne, Bain, Gilfillan, Cecchi,, Stewart & Olstein, 5 Becker Farm Road, Roseland, NJ, 07068, US

(21) Appl. No.: 11/599,845

(22) Filed: Nov. 15, 2006

Related U.S. Application Data
(63) Continuation-in-part of application No. 10/585,466.
(63) Continuation-in-part of application No. 10/583,832.
(63) Continuation-in-part of application No. 10/575,337.
(63) Continuation-in-part of application No. 10/540,310, filed on Dec. 12, 2005.
(63) Continuation-in-part of application No. 10/518,039, filed on Dec. 12, 2005.
(63) Continuation-in-part of application No. 10/516,697, filed on Apr. 27, 2006.
(63) Continuation-in-part of application No. 10/516,476, filed on May 16, 2005.
(63) Continuation-in-part of application No. 10/514,533.
(63) Continuation-in-part of application No. 10/514,534, filed on May 16, 2005.
(63) Continuation-in-part of application No. 10/383,368, filed on Mar. 7, 2003, now abandoned.
(63) Continuation-in-part of application No. 10/379,632, filed on Mar. 5, 2003, now abandoned.
(63) Continuation-in-part of application No. 10/378,973, filed on Mar. 4, 2003, now abandoned.
(60) Provisional application No. 60/531,809, filed on Dec. 22, 2003.
(60) Provisional application No. 60/509,515, filed on Oct. 8, 2003.
(60) Provisional application No. 60/434,960, filed on Dec. 20, 2002.
(60) Provisional application No. 60/388,075, filed on Jun. 11, 2002.
(60) Provisional application No. 60/434,960, filed on Dec. 20, 2002.
(60) Provisional application No. 60/385,505, filed on Jun. 4, 2002.
(60) Provisional application No. 60/380,912, filed on May 16, 2002.
(60) Provisional application No. 60/380,612, filed on May 15, 2002.
(60) Provisional application No. 60/362,527, filed on Mar. 7, 2002.
(60) Provisional application No. 60/362,021, filed on Mar. 6, 2002.
(60) Provisional application No. 60/361,461, filed on Mar. 4, 2002.

(51) Int. Cl. A61K 39/395; A61P 35/00; C07K 16/30; C12Q 1/68

(52) U.S. Cl. 424/138.1; 435/6; 514/789; 530/387.9

(57) ABSTRACT

Cancer-linked gene sequences, and derived amino acid sequences, are disclosed along with methods for assaying for potential antitumor agents based on their modulation of the expression of these cancer-related genes. Also disclosed are antibodies that react with the disclosed polypeptides and methods of using the antibodies to treat cancerous conditions, such as using the antibody to target cancerous cells in vivo for purposes of delivering therapeutic agents thereto. Also described are methods of diagnosing cancer using the gene sequences as well as the polypeptide sequences.

This application is a continuation-in-part of:

U.S. application Ser. No. 10/585,466, which was a national phase filing of PCT/US2005/000040, filed 4 Jan. 2005, which claims priority of U.S. Provisional Application 60/509,515, filed 6 Jan. 2004, and

U.S. application Ser. No. 10/583,832, which was a national phase filing of PCT/US2004/42406, filed 16 Dec. 2004, which claims priority of U.S. Provisional Application 60/531,809, filed 22 Dec. 2003, and

U.S. application Ser. No. 10/575,337, which was a national phase filing of PCT/US2004/33072, filed 7 Oct. 2004, which claims priority of U.S. Provisional Application 60/509,515, filed 8 Oct. 2003, and

U.S. application Ser. No. 10/540,310, which was a national phase filing of PCT/US2003/40710, filed 19 Dec. 2003, which claims priority of U.S. Provisional Application 60/434,960, filed 20 Dec. 2002, and

U.S. application Ser. No. 10/518,039, which was a national phase filing of PCT/US2003/19741, filed 10 Jun. 2003, which claims priority of U.S. Provisional Application 60/388,075, filed 11 Jun. 2002, and

U.S. application Ser. No. 10/516,697, which was a national phase filing of PCT/US2003/17592, filed 5 Jun. 2003, which claims priority of U.S. Provisional Application 60/434,960, filed 20 Dec. 2002, and

U.S. application Ser. No. 10/516,476, which was a national phase filing of PCT/US2003/17559, filed 4 Jun. 2003, which claims priority of U.S. Provisional Application 60/385,505, filed 4 Jun. 2002, and

U.S. application Ser. No. 10/514,533, which was a national phase filing of PCT/US2003/15313, filed 12 May 2003, which claims priority of U.S. Provisional Application 60/380,912, filed 16 May 2002, and

U.S. application Ser. No. 10/514,534, which was a national phase filing of PCT/US2003/15314, filed 15 May 2003, which claims priority of U.S. Provisional Application 60/380,612, filed 15 May 2002, and

U.S. application Ser. No. 10/383,368, filed 7 Mar. 2003, which claims priority of U.S. Provisional Application 60/362,527, filed 7 Mar. 2002, and

U.S. application Ser. No. 10/379,632, filed 5 Mar. 2003, which claims priority of U.S. Provisional Application 60/362,021, filed 6 Mar. 2002,

U.S. application Ser. No. 10/378,973, filed 4 Mar. 2003, which claims priority of U.S. Provisional Application 60/361,461, filed 4 Mar. 2002, and the disclosures of all of which are hereby incorporated by reference in their entirety.

### FIELD OF THE INVENTION

The present invention relates to methods of screening cancer-target genes and expression products for involvement in the cancer initiation and facilitation process and the use of such genes for screening potential anti-cancer agents, including the design of small organic compounds and other molecules, and in the diagnosis of cancer.

### BACKGROUND OF THE INVENTION

Cancer-linked genes are valuable in that they indicate genetic differences between cancer cells and normal cells, such as where a gene is expressed in a cancer cell but not in a non-cancer cell, or where said gene is over-expressed or expressed at a higher level in a cancer as opposed to normal or non-cancer cell. In addition, the expression of such a gene in a normal cell but not in a cancer cell, especially of the same type of tissue, can indicate important functions in the cancerous process. For example, screening assays for novel drugs are based on the response of model cell based systems in vitro to treatment with specific compounds. Various measures of cellular response have been utilized, including the release of cytokines, alterations in cell surface markers, activation of specific enzymes, as well as alterations in ion flux and/or pH. Some such screens rely on specific genes, such as oncogenes (or gene mutations). In accordance with the present invention, cancer-target genes, and encoded polypeptides, have been identified. Such genes are useful in the diagnosing of cancer, the screening of anticancer agents and the treatment of cancer using such agents, especially in that these genes encode polypeptides that can act as markers, such as cell surface markers, thereby providing ready targets for anti-tumor agents such as antibodies, preferably antibodies complexed to cytotoxic agents, including apoptotic agents.

### BRIEF SUMMARY OF THE INVENTION

In one aspect, the present invention relates to a method for identifying an agent that modulates the activity of a cancer-related gene comprising:

(a) contacting a compound with a cell expressing a gene that corresponds to a polynucleotide having a sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768 and under conditions promoting the expression of said gene, preferably where said gene comprises one of said sequences or where said gene encodes the same polypeptide as said sequence, and

(b) determining a difference in expression of said gene relative to when said compound is not present thereby identifying an agent that modulates the activity of a cancer-related gene.

In a preferred embodiment, the cell is a cancer cell and the difference in expression is a decrease in expression.

In another aspect, the present invention relates to a method for determining the cancerous status of a cell, comprising determining an increase in the level of expression in said cell of a gene that corresponds to a one of the foregoing sequences, wherein an elevated expression relative to a known non-cancerous cell indicates a cancerous state or potentially cancerous state. In one embodiment, the gene is a member selected from the group consisting of KIAA1274, NEK6, PAK2, PAK-4, STK38L, ACP1, ARHC, CDC6, CDK7, CDKN3, CRK7, DUSP16, FIGNL1, GUK1, ITPR2, KCNK1, KCNK5, PRO2000, RFC2 and RIPK2. Increased expression may be due to an increased copy number.

In specific examples of these methods, the expression is transcription to form RNA or is translation to form protein.

The present invention also relates to a method for identifying a cancer-target gene, comprising:

a) identifying a gene that is at least 5 fold over-expressed in a cancer cell line and that maps to a chromosomal region with a CGH ratio of at least 1.25;

b) determining an RNA expression level of said gene of at least 1.5 fold in a tumor tissue compared to corresponding normal tissue in a genetic database, and

c) determining that said gene encodes a protein domain that is modulated by chemical compounds,

wherein a gene that meets the criteria of steps a, b and c is considered to be a cancer-target gene,

thereby identifying a cancer-target gene.

The present invention is further drawn to an isolated polypeptide comprising an amino acid sequence homologous to an amino acid sequence selected from the group consisting of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769 wherein any difference between said amino acid sequence and the homologous sequence of said isolated polypeptide is due solely to conservative amino acid substitutions and wherein said isolated polypeptide comprises at least one immunogenic fragment. In a particular embodiment, said polypeptide has one of said sequences.

In other aspects, the present invention encompasses an isolated polypeptide comprising one of said amino acid sequences, an antibody specific for at least one of said a polypeptides and an immunoconjugate comprising a cytotoxic agent and at least one of said antibodies. In non-limiting examples, the cytotoxic agent is a member selected from the group consisting of a calicheamicin (including calicheamicin γ₁^{I}, N-acetyl gamma calicheamicin dimethyl hydrazide or calicheamicin θ₁^{I}), a maytansinoid (e.g., DM1), an adozelesin, a cytotoxic protein (e.g., ricin, abrin, gelonin, pseudomonas exotoxin or diphtheria toxin), a taxol (such as paclitaxel), a taxotere (such as docetaxel), a taxoid or DC1. The invention also includes compositions of said polypeptides, antibodies and/or immunoconjugates, preferably where these are contained in a pharmaceutically acceptable carrier and present in a therapeutically active amount.

The present invention also relates to a method for inhibiting or reducing cancer cell proliferation (or treating cancer) by contacting a cancerous cell in vivo with an agent having activity against an expression product encoded by at least one of the gene sequences disclosed herein, for example, where said agent is an antibody or an immunoconjugate of the invention.

In another aspect, the present invention relates to a method for diagnosing cancer in a patient comprising determining the presence of a cancer-related polypeptide in one or more cells of said patient wherein said cancer-related polypeptide comprises a polypeptide that reacts with an antibody specific for at least one of the polypeptide having an amino acid sequence selected from SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

### DEFINITIONS

As used herein and except as noted otherwise, all terms are defined as given below.

The term “druggable” or “druggable domain” refers to a gene that encodes a protein domain known to be modulated by chemical compounds.

As used herein, the term “isolated” means that the material is removed from its original environment (e.g., the natural environment if it is naturally occurring). It could also be produced recombinantly and subsequently purified. For example, a naturally-occurring polynucleotide or polypeptide present in a living animal is not isolated, but the same polynucleotide or polypeptide, separated from some or all of the coexisting materials in the natural system, is isolated. Such polynucleotides, for example, those prepared recombinantly, could be part of a vector and/or such polynucleotides or polypeptides could be part of a composition, and still be isolated in that such vector or composition is not part of its natural environment. In one embodiment of the present invention, such isolated, or purified, polypeptide is useful in generating antibodies for practicing the invention, or where said antibody is attached to a cytotoxic or cytolytic agent, such as an apoptotic agent.

As known in the art “similarity” between two polypeptides is determined by comparing the amino acid sequence and its conserved amino acid substitutes of one polypeptide to the sequence of a second polypeptide. As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases.

As used herein, the term “corresponding genes” refers to genes that encode an RNA that is at least 90% identical, preferably at least 95% identical, most preferably at least 98% identical, and especially identical, to an RNA encoded by one of the nucleotide sequences of 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768.

As used herein, the term “correspond” also means that the gene has the same nucleotide sequence as a gene identified for use in the methods of the invention or that it encodes substantially the same RNA as would be encoded by the disclosed gene, the term “substantially” meaning at least 90% identical as defined elsewhere herein and includes splice variants thereof or that it encodes the same polypeptide as one of the nucleotide sequences disclosed herein. The methods of the invention may also utilize the complement of any of the disclosed sequences. A “corresponding gene” includes splice variants thereof.

The genes identified by the present disclosure are considered “cancer-related” genes, as this term is used herein, and include genes expressed at higher levels (due, for example, to elevated rates of expression, elevated extent of expression or increased copy number) in cancer cells relative to expression of these genes in normal (i.e., non-cancerous) cells where said cancerous state or status of test cells or tissues has been determined by methods known in the art, such as by reverse transcriptase polymerase chain reaction (RT-PCR) as described in the Examples herein. In specific embodiments, this relates to the genes whose sequences correspond to the polynucleotide sequences disclosed herein.

The term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

The term “expression product” means that polypeptide or protein that is the natural translation product of the gene and any nucleic acid sequence coding equivalents resulting from genetic code degeneracy and thus coding for the same amino acid(s).

As used herein, the term “conservative amino acid substitution” has its usual meaning as described in the literature.

### Sequence Listing on CD-Rom Only

The sequences disclosed herein as SEQ ID NO: 1-769 in the sequence listing are contained on compact disc (CD-ROM) only (File Size: 3.2 Mb), 2 copies of which appear on discs denoted Copy 1 and Copy 2, and which discs were generated on 13 Nov. 2006), which accompanies this application and the contents of said CD-ROMs are hereby incorporated by reference in their entirety.

### DETAILED SUMMARY OF THE INVENTION

The present invention relates to methods for identifying and/or utilizing cancer-related genes, and expression products of such genes, as targets for chemotherapeutic agents, especially anti-cancer agents, and for use in determining the cancerous status of a cell. Genes whose expression, or non-expression, or change in expression, are indicative of the cancerous or non-cancerous status of a given cell and whose expression is changed in cancerous, as compared to non-cancerous cells, from a specific tissue, are genes that are disclosed herein or that are identified by methods disclosed herein. These include genes having structural and/or functional similarity to the genes disclosed herein and include genes that are substantially identical to said genes. In terms of nucleotide sequence, such genes are at least about 90% identical, preferably 95% identical, most preferably at least about 98% identical and especially where such gene comprises a nucleotide sequences disclosed herein.

The cancer-related polynucleotide sequences disclosed herein correspond to gene sequences whose expression is indicative of the cancerous status of a given cell. Such sequences are substantially identical to polynucleotide sequences of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768.

In specific but non-limiting examples, polynucleotides and corresponding polypeptides include the following:

Transcript (no protein encoded): (SEQ ID NO: 110)

Name: T81803_T_{—}1|Length: 2541|Exon numbers: 1, 2

Transcript (SEQ ID NO: 111)

Name: T81803_T_{—}2|Length: 4591|Exon numbers: 4, 5, 6, 7, 8, 9, 10, 11, 12, 13, 14, 15, 18, 19, 20, 21, 22, 23, 24, 25, 26, 27, 28, 29, 30, 32, 33, 34, 35, 36, 37

Protein encoded by transcript 2: (SEQ ID NO: 112)

Name: human_gb124human_{—}224837|Length: 1382|Encoding transcript: 3

Transcript (SEQ ID NO: 113)

Name: T81803_T_{—}3|Length: 4750|Exon numbers: 1, 5, 6, 7, 8, 9, 10, 11, 12, 13, 14, 15, 18, 19, 20, 21, 22, 23, 24, 25, 26, 27, 28, 29, 30, 32, 33, 34, 35, 36, 37

Protein encoded by transcript 3: (SEQ ID NO: 114)

Name: human_gb124human_{—}224837|Length: 1382|Encoding transcript: 3

Transcript (SEQ ID NO: 115)

Name: T81803_T_{—}4|Length: 298|Exon numbers: 17, 18

Protein encoded by transcript 4: (SEQ ID NO: 116)

Name: human_gb124human_{—}172980|Length: 41|Encoding transcript: 4

Transcript (no protein encoded): (SEQ ID NO: 117)

Name: T81803_T_{—}5|Length: 445|Exon numbers: 15, 16

Transcript (SEQ ID NO: 118)

Name: T81803_T_{—}6|Length: 1850|Exon numbers: 26, 27, 28, 29, 30, 31, 32, 33, 34, 35, 36, 37

Protein encoded by transcript 6: (SEQ ID NO: 119)

Name: human_gb124human_{—}172981|Length: 494|Encoding transcript: 6

Transcript (no protein encoded): (SEQ ID NO: 120)

Name: T81803_T_{—}7|Length: 554|Exon numbers: 36, 38, 39

Transcript (SEQ ID NO: 121)

Name: T81803_T_{—}8|Length: 772|Exon numbers: 3, 5, 6, 7

Protein encoded by transcript 8: (SEQ ID NO: 122)

Name: human_gb124human_{—}172982|Length: 2571|Encoding transcript: 8

Transcript (SEQ ID NO: 123)

Name: H53701_T_{—}1|Length: 3559|Exon numbers: 1, 2, 3, 4, 5, 6, 7, 8, 9, 10, 11, 12, 13, 14, 15, 16, 17, 18, 19, 20, 21, 22, 23, 24, 25, 27, 28, 29, 30, 31, 32

Protein encoded by said transcript (SEQ ID NO: 124)

Name: human_gb124human_{—}222520|Length: 1148|Encoding transcript: 1

Transcript (SEQ ID NO: 125)

Name: H53701_T_{—}2|Length: 858|Exon numbers: 26, 28, 29, 30, 31, 32

Protein encoded by said transcript (SEQ ID NO: 126)

Name: human_gb124human_{—}192292|Length: 174|Encoding transcript: 2

Transcript (SEQ ID NO: 127)

Name: H53701_T_{—}3|Length: 372|Exon numbers: 19, 20, 21, 23

Protein encoded by said transcript (SEQ ID NO: 128)

Name: human_gb124human_{—}192293|Length: 104|Encoding transcript: 3

Many of the genes disclosed herein were identified within an amplified chromosomal region in a cancer cell line(s) and exhibit RNA over-expression in the cell line(s) and clinical tumor tissues by Affymetrix microarray analysis. Such genes comprise sequences that encode a protein domain previously described as being modulated by chemical compounds.

Many such genes were identified in a cancer cell line(s) for which high-resolution comparative genomic hybridization (CGH) data and Affymetrix U133 chip expression data were generated and meet the following criteria:
1) At least 5 fold over-expressed in a cancer cell line(s) and mapped to a chromosomal region with a CGH ratio of 1.25 or above.
2) RNA expression level of at least 1.5 fold or higher in tumor tissue samples compared to corresponding normal tissue samples in a genetic database (with Gene Logic GX2000 database being a non-limiting example).
3) The gene encodes a protein domain known to be modulated by chemical compounds (i.e., a “druggable” domain). The genes identified herein represent a subset of all genes in these classes.

In accordance with the foregoing, the present invention also relates to nucleotide sequences and putative encoded polypeptides having the following characteristics (with sequences identified in Table 6):

Arbitrary Gene Name: KIAA1274

Description: KIAA protein (similar to mouse paladin)

UniGene: Hs.300646

Accession: AB033100

Gencarta No. M553584

Cytogenetic location: 10q22.1

Affymetrix fragment: 231887_s_at

Affymetrix ID: 262356

Cell lines involved: SW620

Druggable domain: phosphatase

Arbitrary Gene Name: NEK6

Description: NIMA (never in mitosis gene a)-related kinase 6

UniGene: Hs.9625

Accession: BE616825

Gencarta No. T11445

Cytogenetic location: 9q33.3

Affymetrix fragment: 223158_s_at

Affymetrix ID: 253651

Cell lines involved: Colo205

Druggable domain: kinase

NIMA-related kinases (NEKs) are mammalian serine/threonine protein kinases structurally related to Aspergillus NIMA (never in mitosis, gene A), which play essential roles in mitotic signaling.

Arbitrary Gene Name: PAK2

Description: p21-activated kinase 2

UniGene: Hs.56974

Accession: BF₇₉₆₄₇₀

Gencarta No. Z26993

Cytogenetic location: 3q29

Affymetrix fragment: 208875_s_at

Affymetrix ID: 239505

Cell lines involved: HCC1954, HCC202, HCC70, MDA_MB453, T47D

Druggable domain: kinase

The p21-activated kinases (PAK) are critical effectors that link Rho GTPases to cytoskeleton reorganization and nuclear signaling. The PAK proteins are a family of serine/threonine kinases that serve as targets for the small GTP binding proteins, CDC42 and RAC1, and have been implicated in a wide range of biological activities.

Arbitrary Gene Name: PAK-4

Description: p21-activated kinase 4

UniGene: Hs.20447

Accession: AF005046

Gencarta No. R09837

Cytogenetic location: 19q13.2

Affymetrix fragment: 33814_at

Affymetrix ID: 107335

Cell lines involved: HCC202, MDA_MB468

Druggable domain: kinase

The p21 activated kinases (PAK) are critical effectors that link Rho GTPases to cytoskeleton reorganization and nuclear signaling. The PAK proteins are a family of serine/threonine kinases that serve as targets for the small GTP binding proteins, CDC42 and RAC1, and have been implicated in a wide range of biological activities.

Arbitrary Gene Name: STK38L

Description: serine/threonine kinase 38 like

UniGene: Hs.184523

Accession: AW779556

Gencarta No. R14324

Cytogenetic location: 12p11.23

Affymetrix fragment: 212565_at

Affymetrix ID: 243092

Cell lines involved: HCC827, BEN

Druggable domain: kinase

Arbitrary Gene Name: ACP1

Description: acid phosphatase 1, soluble

UniGene: Hs.75393

Accession: BE872974

Gencarta No. HUMMPA

Cytogenetic location: 2p25.3

Affymetrix fragment: 201629_s_at

Affymetrix ID: 232293

Cell lines involved: BEN, NCl-H460, NCl-H522, MCF7, MDA-MB436, MDA-MB468

Druggable domain: phosphatase

The product of this gene belongs to the phosphotyrosine protein phosphatase family of proteins. It functions as an acid phosphatase and a protein tyrosine phosphatase by hydrolyzing protein tyrosine phosphate to protein tyrosine and orthophosphate. This gene is genetically polymorphic, and three common alleles segregating at the corresponding locus give rise to six phenotypes. Each allele appears to encode at least two electrophoretically different isozymes, Bf and Bs, which are produced in allele-specific ratios. Three transcript variants encoding distinct isoforms have been identified for this gene (Bryson et al., Genomics 1995 Nov. 20; 30(2):133-40).

Arbitrary Gene Name: ARHC

Description: ras homolog gene family, member C (hypothetical protein MGC19531)

UniGene: Hs.446391

Accession: AW117553

Gencarta No. M383349

Cytogenetic location: 1p13.2

Affymetrix fragment: 229484_at

Affymetrix ID: 259953

Cell lines involved: HCC202, MDA_MB468

Druggable domain: phosphatase

ARHC (UniGene Hs.179735) sits next to the gene for hypothetical protein MGC19531. The two sequences are in close proximity and they are annotated as the same gene in GenCarta, but they are listed as two distinct genes in UCSC Goldenpath. The Affymetrix fragment maps within the sequence for hypothetical protein MGC19531, but we are inferring that this fragment is detecting expression for ARHC.

ARHC encodes a ras-related GTP binding protein of the rho subfamily, member C (RhoC) that regulates remodeling of the actin cytoskeleton during cell morphogenesis and motility. Up regulation of RhoC through increased expression of ARHC has been reported in breast, ovarian and pancreatic cancer as well as melanoma and has been associated with progression to a metastatic phenotype in each cancer type (van Golen et al., Cancer Res. 2000 Oct. 15; 60(20):5832-8, Horiuchi A et al. Lab Invest. 2003 83(6):861-70, Suwa et al. Br J. Cancer. 1998 77(1):147-52, Clark et al., Nature, 2000 406(6795):532-5).

Arbitrary Gene Name: CDC6

Description: CDC6 cell division cycle 6 homolog (S. cerivisiae)

UniGene: Hs.69563

Accession: U77949

Gencarta No. T83032

Cytogenetic location: 17q21.3

Affymetrix fragment: 203967_at

Affymetrix ID: 234629

Cell lines involved: NCl_H522, NCl_H23

Druggable domain: AAA ATPase

Yan et al. [Proc Nat Acad Sci 94:142-147 (1998)] showed that CDC6 is expressed selectively in proliferating but not quiescent mammalian cells, both in culture and within tissues in intact animals. During the transition from a growth-arrested to a proliferative state, transcription of mammalian CDC6 is regulated by E2F proteins as revealed by a functional analysis of the promoter and by the ability of exogenously expressed E2F proteins to stimulate endogenous CDC6. Immunodepletion of CDC6 protein by microinjection of anti-CDC6 antibody blocked initiation of DNA replication in a human tumor cell line. The authors concluded that expression of human CDC6 is regulated in response to mitogenic signals through transcriptional control mechanisms involving E2F proteins, and that CDC6 protein is required for initiation of DNA replication in mammalian cells.

Arbitrary Gene Name: CDK7

Description: Cyclin-dependent kinase 7 (MO15 homolog, Xenopus laevis)

UniGene: Hs.184298

Accession: X77743

Gencarta No. F02366

Cytogenetic location: 5q13.2

Affymetrix fragment: 211297_s_at

Affymetrix ID: 241855

Cell lines involved: SW620

Druggable protein domain: kinase

The protein encoded by CDK7 is a member of the cyclin-dependent protein kinase (CDK) family, which are known to be important regulators of cell cycle progression. This protein forms a trimeric complex with cyclin H and MAT1, which functions as a Cdk-activating kinase (CAK) (Fisher and Morgan, Cell 78:713-724, 1994). It is an essential component of the transcription factor IIH (TFIIH) that is involved in transcription initiation and DNA repair (Shiekhattar et al., Nature 374: 283-287, 1995). This protein is thought to serve as a direct link between the regulation of transcription and the cell cycle.

Arbitrary Gene Name: CDKN3

Description: cyclin-dependent kinase inhibitor 3 (CDK2-associated dual specificity phosphatase)

UniGene: Hs.84113

Accession: AF213033

Gencarta No. HUMPTPB

Cytogenetic location: 14q22.2

Affymetrix fragment: 209714_s_at

Affymetrix ID: 240337

Cell lines involved: NCl-H460, HCC827, NCl-H23, HCC202, MCF7, MDA-MB453, T47D

Druggable domain: phosphatase

The protein encoded by this gene is a human dual specificity protein phosphatase that was identified as a cyclin-dependent kinase inhibitor, and has been shown to interact with and dephosphorylate CDK2 kinase and thus prevent the activation of CDK2 kinase. The gene has been reported to be deleted, mutated, or overexpressed in several kinds of cancers.

Lee et al. [Mol Cell Biol. 2000 March; 20(5):1723-32} identified the CDKN3 as an overexpressed gene in breast and prostate cancer by using a phosphatase domain-specific differential-display PCR strategy. They report in normal cells, CDKN3 protein is primarily found in the perinuclear region, but in tumor cells, a significant portion of the protein is found in the cytoplasm. Blocking CDKN3 expression by antisense in a tetracycline-regulatable system resulted in a reduced population of S-phase cells and reduced Cdk2 kinase activity. Furthermore, lowering CDKN3 expression led to inhibition of the transformed phenotype, with reduced anchorage-independent growth and tumorigenic potential in athymic nude mice. They suggest that therapeutic intervention might be aimed at repression of CDKN3 gene overexpression in human breast and prostate cancer.

Yeh et al. [Cancer Res. 1;60(17):4697-4700 (2000)] analyzed CDKN3 mRNA in hepatocellular carcinoma by reverse transcription-PCR(RT-PCR), followed by cloning and sequencing. They found aberrant CDKN3 transcripts in hepatocellular tumors and showed mutant proteins were defective in interacting with Cdk2.

Arbitrary Gene Name: CRK7

Description: CDC2-related protein kinase 7

UniGene: Hs.278346

Accession: A1651265

Gencarta No. T60764

Cytogenetic location: 17q12

Affymetrix fragment: 225697_at

Affymetrix ID: 256169

Cell lines involved: HCC1954, HCC202, SKBR3

Druggable domain: kinase

Ko et al. [Journal of Cell Science, 114,2591-2603 (2001)] isolated and characterized CrkRS, CDC2-related kinase 7, as a novel human protein with an arginine/serine-rich (RS) domain that is most closely related to the cyclin-dependent kinase family. They report CrkRS is a 1490 amino acid protein where the protein kinase domain is 89% identical to CHED protein kinase. CrkRS has extensive proline-rich regions that match the consensus for SH3 and WW domain binding sites and RS domain that is predominantly found in splicing factors. The authors describe CrkRS as a novel, conserved link between the transcription and splicing machinery of a cell.

Arbitrary Gene Name: DUSP16

Description: dual specificity phosphatase 16

UniGene: Hs.20281

Accession: AB051487

Gencarta No. T23935

Cytogenetic location: 12p13.2

Affymetrix fragment: 224832_at

Affymetrix ID: 255305

Cell lines involved: HCC827

Druggable domain: phosphatase

Mitogen-activated protein kinase (MAPK) phosphatases (MKPs) negatively regulate MAPK activity. DUSP16 is a dual specificity phosphatase that functions as a MAPK phosphatase, also known as MKP7. Masuda et al. [J Biol. Chem. 2001 276(42):39002-[1] showed that MAPK7 behaves as a nuclear shuttle for c-Jun terminal kinase (JNK) group of MAPKs as well as a phosphatase.

Arbitrary Gene Name: FIGNL1

Description: fidgetin-like 1

UniGene: Hs.137516

Accession: M805691

Gencarta No. H61320

Cytogenetic location: 7p12.2

Affymetrix fragment: 222843_at

Affymetrix ID: 253337

Cell lines involved: SW620, BEN, HCC827

Druggable domain: AAA ATPase

Arbitrary Gene Name: GUK1

Description: guanylate kinase 1

UniGene: Hs.3764

Accession: BC006249

Gencarta No. T08090

Cytogenetic location: 1q42.13

Affymetrix fragment: 200075_s_at

Affymetrix ID: 231232

Cell lines involved: HCC202, MDA_MB436, MDA_MB453, MDA_MB468

Druggable domain: kinase

Guanylate kinase catalyzes the phosphorylation of either GMP to GDP or dGMP to dGDP and is an essential enzyme in nucleotide metabolism pathways. There are several isoforms, GUK2 and GUK3, determined by different loci. Brady et al. [J Biol. Chem. 1996 12;271(28):16734-40] stated that the guanylate kinases are targets for cancer chemotherapy and are inhibited by the drug 6-thioguanine. They report a model of the tertiary structure designed to be used in the development of chemotherapy drugs.

Arbitrary Gene Name: ITPR2

Description: inositol 1,4,5-triphosphate receptor, type 2

UniGene

Accession: D26350

Gencarta No. Z38709

Cytogenetic location: 12p11.23

Affymetrix fragment: 211360_s_at

Affymetrix ID: 241911

Cell lines involved: BEN, HCC827

Druggable domain: Ion transport

Arbitrary Gene Name: KCNK1

Description: potassium channel, subfamily K, member 1

UniGene: Hs.79351

Accession: U90065

Gencarta No. Z39663

Cytogenetic location: 1q42.2

Affymetrix fragment: 204678_s_at

Affymetrix ID: 235340

Cell lines involved: HCC202, HCC70, MDA_MB436, MDA_MB453, MDA_MB468

Druggable domain: potassium channel

This gene encodes one of the members of the superfamily of potassium channel proteins containing two pore-forming P domains and 4 transmembrane segments. Potassium channels are functionally important to a large number of cellular processes including maintenance of the action potential, muscle contraction, hormone secretion, osmotic regulation and ion flow.

Arbitrary Gene Name: KCNK5

Description: potassium channel, subfamily K, member 5

UniGene: Hs.127007

Accession: AI678413

Gencarta No. R25184

Cytogenetic location: 6p21.2

Affymetrix fragment: 69854_at

Affymetrix ID: 153971

Cell lines involved: Colo201, Colo205

Druggable domain: K+ channel

This gene encodes one of the members of the superfamily of potassium channel proteins containing two pore-forming P domains.

Arbitrary Gene Name: PRO2000

Description: Hypothetical protein MGC5254

UniGene: Hs.222088

Accession: AI925583

Gencarta No. Z44462

Cytogenetic location: 8q24.13

Affymetrix fragment: 222740_at

Affymetrix ID: 253234

Cell lines involved: BT549, HCC1954, HCC202, HCC70, Hs578t, MCF7, MDA_MB231, MDA_MB436, MDA_MB453, SKBR3, T47D, Colo201, HCT116, SW620, HT29, HCC827, NCl-H23, NCl-H460

Druggable domain: AAA ATPase

A large family of ATPases has been described, whose key feature is that they share a conserved region of about 220 amino acids that contains an ATP-binding site. The protein encoded by PRO2000 contains two AAA (ATPases Associated with diverse cellular Activities) domains as well as a bromodomain. AAA family proteins often perform chaperone-like functions that assist in the assembly, operation, or disassembly of protein complexes. The exact function of the PRO2000 protein is unknown.

Fellenberg et al. [Int J Cancer 105(5);636-643 (2003)] report PRO2000 is up-regulated >2 fold in osteosarcoma cell line (Saos-2) following treatment with cisplatin, methotrexate and doxorubicin.

ARBITRARY Gene Name: RFC2

Description: replication factor C (activator 1) 2, 40 kDa

UniGene: Hs.139226

Accession: M87338

Gencarta No. T62520

Cytogenetic location: 7q11.23

Affymetrix fragment: 1053_at

Affymetrix ID: 113880

Cell lines involved: HCC827, NCl_H23, NCl_H522

Druggable domain: AAA ATPase

The elongation of primed DNA templates by DNA polymerase delta and epsilon requires the action of the accessory proteins proliferating cell nuclear antigen (PCNA) and replication factor C(RFC). RFC, also called activator 1, is a protein complex consisting of five distinct subunits of 145, 40, 38, 37, and 36.5 kD. This gene encodes the 40 kD subunit, which has been shown to be responsible for binding ATP. Alternatively spliced transcript variants encoding distinct isoforms have been described.

Arbitrary Gene Name: RIPK2

Description: receptor-interacting serine-threonine kinase 2

UniGene: Hs.103755

Accession: AF064824

Gencarta No. D61791

Cytogenetic location: 8q21.3

Affymetrix fragment: 209545_s_at

Affymetrix ID: 240173

Cell lines involved: HCT116

Druggable domain: kinase

TABLE 1
Brief summary of above target genes (GenBank Accession Nos.)

| Accession | unigene | affy | Description |
|---|---|---|---|
| AI651265 | Hs.278346 | 256169 | CDC2-related protein kinase 7 |
| U77949 | Hs.69563 | 234629 | CDC6 cell division cycle 6 homolog (S. |
| | | | cerevisiae) |
| X77743 | Hs.184298 | 241855 | cyclin-dependent kinase 7 (MO15 homolog, |
| | | | Xenopus laevis, cdk-activating kinase) |
| AA805691 | Hs.137516 | 253337 | fidgetin-like 1 |
| AI925583 | Hs.222088 | 253234 | hypothetical protein MGC5254 |
| D26350 | | 241911 | inositol 1,4,5-triphosphate receptor, type 2 |
| | Hs.406293 | 253470 | neurotrophic tyrosine kinase, receptor, type 1 |
| BE616825 | Hs.9625 | 253651 | NIMA (never in mitosis gene a)-related |
| | | | kinase 6 |
| BF796470 | Hs.56974 | 239505 | p21 (CDKN1A)-activated kinase 2 |
| AF005046 | Hs.20447 | 107335 | p21 (CDKN1A)-activated kinase 4 |
| U90065 | Hs.79351 | 235340 | potassium channel, subfamily K, member 1 |
| AF064824 | Hs.103755 | 240173 | receptor-interacting serine-threonine kinase 2 |
| M87338 | Hs.139226 | 113180 | replication factor C (activator 1) 2, 40kDa |
| AW779556 | Hs.184523 | 243092 | serine/threonine kinase 38 like |
| AI678413 | Hs.127007 | 153971 | potassium channel, subfamily K, member 5 |
| AF213033 | Hs.84113 | 240337 | cyclin-dependent kinase inhibitor 3 (CDK2- |
| | | | associated dual specificity phosphatase) |
| BE872974 | Hs.75393 | 232293 | acid phosphatase 1, soluble |
| AW117553 | Hs.446391 | 259953 | hypothetical protein MGC19531 (ras |
| | | | homolog gene family, member C) |
| BC006249 | Hs.3764 | 231232 | guanylate kinase 1 |
| AB033100 | Hs.300646 | 262356 | KIAA protein (similar to mouse paladin) |
| AB051487 | Hs.20281 | 255305 | dual specificity phosphatase 16 |

TABLE 2
Chromosome Location of the above Cancer Target Genes

| Accession | Chromosome | band | Description | indication | Primer |
|---|---|---|---|---|---|
| AI651265 | chr17 | q12 | kinase | | PR3869 |
| U77949 | chr17 | q21.2 | AAA ATPase | breast | PR3870 |
| X77743 | chr5 | q13.2 | kinase | ovary | PR3871 |
| AA805691 | chr7 | p12.2 | AAA ATPase | lung | PR3872 |
| AI925583 | chr8 | q24.13 | AAA ATPase | | PR3873 |
| D26350 | chr12 | p11.23 | ion transport | | PR3874 |
| | chr1 | q21.3 | tk | melanoma and | PR3875 |
| | | | | LN mets | |
| BE616825 | chr9 | q33.3 | kinase | sarcoma | PR3876 |
| BF796470 | chr3 | q29 | kinase | ovary | PR3877 |
| AF005046 | chr19 | q13.2 | kinase | | PR3878 |
| U90065 | chr1 | q42.2 | K+ channel | pancreas | PR3879 |
| AF064824 | chr8 | q21.3 | kinase | ovary | PR3880 |
| M87338 | chr7 | q11.23 | AAA ATPase | | PR3881 |
| AW779556 | chr12 | p11.23 | kinase | pancreas | PR3882 |
| AI678413 | chr6 | p21.2 | K+ channel | | PR3883 |
| AF213033 | chr14 | q22.2 | | | PR3884 |
| BE872974 | chr2 | p25.3 | | | PR3885 |
| AW117553 | chr1 | p13.2 | Phosphatase | breast | PR3886 |
| BC006249 | chr1 | q42.13 | | | PR3887 |
| AB033100 | chr10 | q22.1 | | | PR3888 |
| AB051487 | chr12 | p13.2 | | | PR3889 |

The methods of the invention utilize these genes, designated as KIAA1274, NEK6, PAK2, PAK4, STK38L, ACP1, ARHC, CDC6, CDK7, CDKN3, CRK7, DUSP16, FIGNL1, GUK1, ITPR2, KCNK1, KCNK5, PRO2000, RFC2 and RIPK2.

Table 2 describes the location of the cancer target genes of the present invention while Table 3 describes primers used to locate these genes. An additional set of primers is provided in Table 5 while additional gene data is provided in Table 4. The nucleotides and polypeptides, as gene products, used in the methods of the present invention may comprise a recombinant polynucleotide or polypeptide, a natural polynucleotide or polypeptide, or a synthetic polynucleotide or polypeptide, preferably a recombinant polynucleotide or polypeptide.

TABLE 3
Primers used to Identify Genes

| | | |
|---|---|---|
| Accession | Left primer 1 | Right primer 1 |

| | | |
|---|---|---|
| A1651265 | TCTTGGGCATCTCAACG | CACATAAAGCAGGTTGTAG |
| | | AACG |
| U77949 | TATTCAGCTGGCATTTAGAGA | ACACTTGCAAGCACATTGG |
| | GC | C |
| X77743 | TGGACAACATTTTACTACTGA | TAAGTTTTCCCTAATGCAT |
| | GGG | TTTCA |
| AA805691 | TTAGTCCTGATAAAAATTAAA | AGAAAGTGCCTTCCTGATG |
| | AACC | |
| AI925583 | TAAACCATTTGGGATGGCAT | TGGAACAAGCTGTTAACAC |
| | | CC |
| D26350 | CTCTGAGGACATTCCCGTTAG | CAGGTGTTTCAAGGAAGAG |
| | AAATGACATGGGGCTTGC | GAAAACACCTGAGGGGGCT |
| | | T |
| BE616825 | TCTTCATGAATTCTAAGTAAC | GTCACTGACTTCATGACA |
| | TC | |
| BF796470 | TAACAAGCGATTCTAAACCAC | ATGGATGCAAATTCTTTAA |
| | C | GCA |
| AF005046 | AACTAACTCGAGGCAGGGGT | CTGCCCTTATTGGGGGAC |
| U90065 | TTCCCCTTATTTTATTGTAGC | GGTTTATGTGTACTGGTTT |
| | AA | GCA |
| AF064824 | AAATGGGGACAGGAAGCC | GCTTAATTGCCCTACAAAG |
| | | GG |
| M87338 | CAACAAACACTGCAAGGCTT | TCTCCATCCTGGGGAAAAA |
| AW779556 | TGCCACCAAAACATTTTTGA | ATGTGAGGGGATATTGCTG |
| | | C |
| AI678413 | AACTACTACACACAGAAGCTG | AAGCCAGCTTCAGATGTAT |
| | C | AT |
| AF213033 | CCATGTCTGAAATGTCAGTTC | AAAACTTTAGGAATATCTG |
| | TC | CACATG |
| BE872974 | TCAGAGGCAAAGTGGTTCAG | AATCAGTCGTTGGCACCTT |
| | | C |
| AW117553 | TCTTGACACATACGAAGCC | GTAGAAGCAGAGTCCCTGG |
| BC006249 | AGGCTTGCTGTCTGTTCTCG | TTTATTAGGATGTCAGCCC |
| | | TGG |
| AB033100 | CTTCTCCTCAGTCTCAAACCC | ATCCATCTCTCTGACAGTG |
| | AA | CTGA |
| AB051487 | ATCCCATTTTAAACAATTCTT | GCTGAACCACCAGGAACCT |
| | TGA | |

TABLE 4
Further Description of Cancer Target Genes

| | | | | Gencarta |
|---|---|---|---|---|
| Accession | | | UniSTS | Name |
| AI651265 | PR3890 | CRK7 | SHGC-58832 | T60764 |
| U77949 | PR3891 | CDC6 | RH70424 | T83032 |
| X77743 | PR3892 | CDK7 | SHGC-149358 | F02366 |
| AA805691 | PR3893 | FIGNL1 | RH103568 | H61320 |
| AI925583 | PR3894 | PRO2000 | RH80934 | Z44462 |
| D26350 | PR3895 | ITPR2 | SHGC-106565 | Z38709 |
| | PR3896 | | SHGC-69193 | |
| BE616825 | PR3897 | NEK6 | RH62928 | T11445 |
| BF796470 | PR3898 | PAK2 | SHGC-35416 | Z26993 |
| AF005046 | PR3899 | PAK4 | RH39107 | R09837 |
| U90065 | PR3900 | KCNK1 | 55164 | Z39663 |
| AF064824 | | RIPK2 | | D61791 |
| M87338 | PR3901 | RFC2 | 47404 | T62520 |
| AW779556 | PR3902 | STK38L | 182659 | R14324 |
| AI678413 | PR3903 | KCNK5 | 83108 | R25184 |
| AF213033 | PR3904 | CDKN3 | 24341 | HUMPTPB |
| BE872974 | PR3905 | ACP1 | 91295 | HUMAAPA |
| AW117553 | PR3906 | ARHC | RH49960 | AA383349 |
| BC006249 | PR3907 | GUK1 | 38548 | T08090 |
| AB033100 | PR3908 | KIAA1274 | 148013 | AA553584 |
| AB051487 | PR3909 | DUSP16 | 85676 | T23935 |

TABLE 5
Additional Primers for Cancer Target Genes

| | | |
|---|---|---|
| Accession | Left primer 2 | Right primer 2 |

| | | |
|---|---|---|
| A1651265 | GTGGGGCCCAATAACTCAAA | TTTTGAATCTGGCCTTGCC |
| | | T |
| U77949 | TTATGACCCCAACGCC | AAGCAAGTCCACATGGAG |
| X77743 | CAGAGGTTCCCTCTTAAAAAT | AAAGTGAAGTATTGGCTGG |
| | TCA | GC |
| AA805691 | CCATCCATGGAATCCTAGACA | TTATCCTACCACTTTGCGG |
| | | G |
| AI925583 | AAGAGTTGGCCAAACTTCAAC | TGTCATGTCCGCCTAATTG |
| | TATT | A |
| D26350 | CAAAGCCTCAAGACCTTTTTC | AAGGTACCAGCTAAACCTC |
| | AAGAGAAAGGGAGGGATCGTT | TTTGCTGTGAGGGGCTATG |
| | C | CTGG |
| BE616825 | TTCCACTTTATCCCTTTACAA | GGCTTATGCTAACAGGAGA |
| | CA | CTTG |
| BF796470 | TCACTGCTGTGGCCTCATAC | TCAGTCCACAATTCCTTCT |
| | | GG |
| AF005046 | GGGGGACGCTGTCATTCAC | TTCCCAGTACCGCAGAGCC |
| U90065 | GGTCCTCTACTTCCACAT | GCTCTCTGAATTTTTGATT |
| AF064824 | | |
| M87338 | GCAGAGACTTCACTGACTGAC | TGACCTCAGGTGATCCACC |
| | | TG |
| AW779556 | TTTAGCAAAACTTGGAGCTGG | AAAACCATTCTCTACTAAC |
| | AG | TACCCCC |
| AI678413 | TTTTGCAAGGCAACTGAGG | GATACGGCAGCCTCTACTGC |
| AF213033 | CACATGGCCTAGTAGTTTGG | GTTCCAACTGCTTAGATCAG |
| | | C |
| BE872974 | TGAACAAAGAGCTGGGCTTT | ACTGAGGCAGGTTCGTGC |
| AW117553 | AGCCTGTAGCCTTTATCCATG | CTTCTGGCTCACAGGAAAAT |
| | | G |
| BC006249 | CTGCTCTTTACCTGGGGTTG | GAGCCACAGAGGAGTGAAGG |
| AB033100 | ACATGTGCCCTACACACAC | AGCTGTCACATAAATAGAAC |
| | | CC |
| AB051487 | ATCAGACATTCTCAAGTTTCA | GGACCATGGCCAAGAGAAG |
| | CACA | |

Table 6 below contains a listing of the genes (numbered 1 to 20) along with their Gencarta names (or accessions). Each gene is represented as a consensus sequence followed by predicted mRNA transcripts and then predicted polypeptides. All of the sequences, with additional information, are presented in FIG. 1.

The present invention also relates to an isolated cancer target gene wherein said gene is a gene identified in Table 6. Thus, the present invention encompasses isolated genes identified herein as KIAA1274, NEK6, PAK2, PAK-4, STK38L, ACP1, ARHC, CDC6, CDK7, CDKN3, CRK7, DUSP16, FIGNL1, GUK1, ITPR2, KCNK1, KCNK5, PRO2000, RFC2 and RIPK2 and uses of these genes, whether isolated or not, in any of the methods of the invention.

TABLE 6
Sequence Identification Numbers for genes, transcripts and
polypeptides*

| Gene | Accession No. | Consensus | Transcripts | Polypeptides |
|---|---|---|---|---|
| ARHC | AA383349 | 195 | 196-204 | 205-210 |
| KIAA1274 | AA553584 | 211 | 212-216 | 217-221 |
| RIPK2 | D61791 | 222 | 223-230 | 231-234 |
| CDK7 | F02366 | 235 | 236-250 | 251-262 |
| FIGNL1 | H61320 | 263 | 264-269 | 270-272 |
| ACP1 | HUMAAPA | 273 | 274-298 | 299-309 |
| CDKN3 | HUMPTPB | 310 | 311-319 | 320-325 |
| PAK4 | R03897 | 326 | 327-344 | 345-352 |
| STK38L | R14324 | 353 | 354-359 | 360-364 |
| KCNK5 | R25184 | 365 | 366-370 | 371-373 |
| GUK1 | T08090 | 374 | 375-429 | 430-448 |
| NEK6 | T11445 | 449 | 450-476 | 477-488 |
| DUSP16 | T23935 | 489 | 490-504 | 505-509 |
| CRK7 | T60764 | 510 | 511-523 | 524-528 |
| RFC2 | T62520 | 529 | 530-559 | 560-572 |
| CDC6 | T83032 | 573 | 574-583 | 584-586 |
| PAK2 | Z26993 | 587 | 588-609 | 610-615 |
| ITPR2 | Z38709 | 616 | 617-633 | 634-643 |
| KCNK1 | Z39663 | 644 | 645-655 | 656-664 |
| PRO2000 | Z44462 | 665 | 666-684 | 685-695 |

*Accession numbers in Table 6 are for the Gencarta database.

In a GX2000 screen for genes specifically expressed in prostate tissue the following ESTs were identified. The Gencarta program or custom EST clustering or Unigene clusters elongated these sequences and predicted protein coding capacity of these elongated sequences. Some of these sequences represent ‘known’ sequences that are available as ‘hypothetical’ or ‘known’ proteins in the database. Where indicated, these sequences represent transmembrane proteins. These are especially useful as diagnostic markers, for screening purposes and treatment.

The genes useful in the methods of the present invention (along with GenBank accession numbers) also include the following:

1. BC006219 (SMP1, Unigene cluster Hs.447531), coding for BC006219, similar to mesoderm posterior 1 (MESP1), also expressed in malignant endometrium. This corresponds to SEQ ID NO: 701, encoding the polypeptide with amino acid sequence of SEQ ID NO: 702.

2. AW245867 (updated as AW137133; Unigene cluster Hs.163113, updated as Hs.245867), is extended by the Gencarta program that predicts 4 different splice variants, all of them with protein coding capacity. SAGE data for the splice variants suggests expression preferentially in prostate. The variants include SEQ ID NO: 703, 704, 706, 708 and 710, which encode polypeptides with SEQ ID NO: 704, 707, 709 and 711.

3. AB059429 (Unigene cluster Hs.306812, SEQ ID NO: 712) coding for protein BAB64535 ‘Butyryl-CoA-Synthetase 1’ (SEQ ID NO: 713). Expressed selectively in prostate cancer, virtually no expression in nonmalignant tissue.

4. BC001186 (Unigene cluster Hs.119693, SEQ ID NO: 714) coding for protocadherin beta 5 (PCDHB5, SEQ ID NO: 715, see BAB64535) Transmembrane protein.

5. AI741645 (Unigene cluster Hs.125350, SEQ ID NO: 716) sequence is extended by Gencarta program that predicts to alternatively spliced forms (SEQ ID NO: 717 and 719), one of them (SEQ ID NO: 717) with protein (SEQ ID NO: 718) coding capacity.

6. AL043002 (Unigene cluster Hs.469918, SEQ ID NO: 720), sequence is identical to AL136789 (Unigene cluster Hs.469927, SEQ ID NO: 721), nucleotide sequence for ‘hypothetical protein dkfz p434F1719’ with amino acid sequence CAB66723 (SEQ ID NO: 722).

7. BC004992 (Unigene cluster Hs. 148322, SEQ ID NO: 723) coding for AAH04992 ‘hypothetical protein MGC4400’ (SEQ ID NO: 724) and CAB66723. The protein contains a ‘KRAB’ motive for transcriptional repression and 8 Zinc finger domains for DNA binding. Expression is largely prostate cancer specific, i.e. no expression in normal prostate tissue and BPH.

8. AI953589 (Unigene cluster Hs.373533, updated Hs.145251, SEQ ID NO: 725) coding for the protein ‘Mirror-image polydactyly 1(MIPOL1)’. Gencarta predicts 10 splice variants with protein coding capacity, variant 3 representing the actual published MIPOL1. Only variants 1, 4, 5, 6, and 7 contain the EST sequence AI953589 (updated as AAH04992, zinc finger protein 577 (ZNF577) and represent prostate specific transcripts. Variants 4 (SEQ ID NO: 728, encoding the polypeptide of SEQ ID NO: 729), variant 5 (SEQ ID NO: 730, encoding the polypeptide of SEQ ID NO: 731), variant 6 (SEQ ID NO: 732, encoding the polypeptide of SEQ ID NO: 733), and variant 7 (SEQ ID NO: 734, encoding the polypeptide of SEQ ID NO: 735) lead to the same protein as variant 3 (MIPOL1) and can be selectively targeted by RNAi because of their unique 3′-ends. Variant 1 (SEQ ID NO: 726, encoding the polypeptide of SEQ ID NO: 727) displays a unique N-terminus on the polypeptide (with the unique peptide HAATTLLILHRYRLNEGFLLGLDP), representing a prostate specific antigen. The variants also encode polypeptides of SEQ ID NO: 729, 729, 731, 733 and 735.

9. NM_{—}024788, (Unigene cluster Hs.521012) with splice variant 1 (SEQ ID NO: 736, encoding the polypeptide of SEQ ID NO: 737, hypothetical protein FLJ), variant 2 (SEQ ID NO: 738, encoding the polypeptide of SEQ ID NO: 739), variant 3 (SEQ ID NO: 740, encoding the polypeptide of SEQ ID NO: 741), and variant 4 (SEQ ID NO: 742, encoding the polypeptide of SEQ ID NO: 743).

10. BF447038 (Unigene cluster Hs.195992, SEQ ID NO: 744) and AK056857 (SEQ ID NO: 745) and polypeptide (SEQ ID NO: 746, BAB71297).

11. AW167298 (Unigene cluster Hs.534881, SEQ ID NO: 747) including splice variant 1 (SEQ ID NO: 748, encoding the polypeptide of SEQ ID NO: 749), variant 2 (SEQ ID NO: 750), variant 3 (SEQ ID NO: 751) and variant 4 (SEQ ID NO: 752, encoding the polypeptide of SEQ ID NO: 753).

12. BG032839 (Unigene cluster Hs.530660, SEQ ID NO: 754, encoding the polypeptide of SEQ ID NO: 755).

13. AL450314, AL589866 and AL590118, (Unigene cluster Hs.360940) with splice variants encoding a serine hydrolase-like protein, including variant 1 (SEQ ID NO: 756, encoding the polypeptide of SEQ ID NO: 757), variant 2 (SEQ ID NO: 758, encoding the polypeptide of SEQ ID NO: 759), variant 3 (SEQ ID NO: 760, encoding the polypeptide of SEQ ID NO: 761) and variant 4 (SEQ ID NO: 762, encoding the polypeptide of SEQ ID NO: 763).

14. NM_{—}023938 (Unigene cluster Hs.223394, updated Hs.32417, SEQ ID NO: 764) coding for a ‘hypothetical protein MGC2742’ (SEQ ID NO: 766).

The corresponding cDNA is SEQ ID NO: 765.

15. AL590118 (Unigene cluster Hs.348556, updated Hs.360940, SEQ ID NO: 767) coding for a hypothetical protein CGI-96 (SEQ ID NO: 769, for which the corresponding cDNA is SEQ ID NO: 768).

Some cDNAs of the invention (for example, SEQ ID NO: 697 or 699 from prostate-specific mRNA) are useful for screening for disseminated tumor cells by PCR, and immunotherapy of prostate cancer using transfected antigen-presenting cells. Proteins of the invention (SEQ ID NO: 698 or 700) are useful as a prostate specific antigen, for screening for disseminated tumor cells by antibody, and for immunotherapy of prostate cancer using protein or related peptides for induction of CD4+ cells and CD8+ cells. These sequences represent new prostate-specific transcripts that are expressed in virtually all prostate cancers. Fragments of these sequences have been described as being prostate-specific by using a mere in silico approach (see Walker, M. G. et al., Prediction of gene function by genome-scale expression analysis: prostate cancer-associated genes. Genome Res. 9: 1198-1203 (1999)). However, in accordance with the present invention, these sequences were detected in a GX2000 database search and extended. Experimental data for their tissue specificity was demonstrated. Here, SEQ ID NO: 696 has accession AF109300. SEQ ID NO: 699 and 700 represent splice variants.

In a GX2000 screen for Genes specifically expressed in prostate tissue the EST AF109300 (SEQ ID NO: 696) was identified. Electronic northern provided evidence for expression in about 50% of all normal and malignant prostate tissue samples in the GX2000 database. Background expression in all available normal tissues was undetectable. AF109300 is part of a known cDNA library (Incyte Pharmaceuticals) and is called D-PCa-3 (prostate cancer associated protein). The prostate-cancer association was determined by an in silico method called “guilt-by-association” (see Walker et al, supra).

The sequence was also identified in a GX2000 screen and provided experimental evidence for prostate-specific expression by hybridyzation of a multiple tissue expression array (from BD Clontech) with a radioactive probe representing AF109300. Prostate-specific expression was also demonstrated by quantitative PCR using a human cDNA panel (from BD Clontech) containing cDNA of 16 major organs as templates. Moreover, the sequence was extended using 5′- and 3′-RACE (rapid amplification of cDNA ends). The subsequently identified open reading frame comprises nucleotides coding for a putative protein containing 7 predicted transmembrane domains and a truncated DUF590 domain. A secreting signal is lacking. However, the ORF is still open at the 5′-end, indicating that the sequence is incomplete at its 5′-end. The present application also discloses a splice variant.

In accordance with the present invention, CYP4Z1 is a new breast-specific transcript that is expressed in female breast tissue and is upregulated in breast cancer. It encodes a protein belonging to the family of cytochrome P450 oxidases and is therefore predicted to have enzymatic activity. Because of its highly specific expression in female breast tissue, it is a useful target for cell-based anti-cancer immunotherapy. This polypeptide has the sequence shown in SEQ ID NO: 194, with the corresponding cDNA shown as SEQ ID NO: 193.

The present application discloses polynucleotides (including two known EST sequences with Accession Numbers A1668602 (SEQ ID NO: 191) and AV700083—SEQ ID NO: 192) and a polypeptide (CYP4Z1—SEQ ID NO: 194) with its encoding cDNA transcript (SEQ ID NO: 193) useful in the methods of the invention.

CYP4Z1 cDNA (SEQ ID NO: 193 from breast-specific mRNA) is useful for screening for disseminated tumor cells by PCR, and immunotherapy of breast cancer using transfected antigen-presenting cells. CYP4Z1 protein (SEQ ID NO: 194) is useful as a breast specific antigen, for screening for disseminated tumor cells by antibody, and for immunotherapy of breast cancer using protein or related peptides for induction of CD4+ cells and CD8+ cells. The expression and activity of this polypeptide, as an indicator of the cancerous state and for screening for antitumor agents, can be determined by a measure of P450 oxidase activity.

In a GX2000 screen for genes specifically expressed in female breast tissue the EST AI668602 (95 k chip) was identified. Supporting this finding, the EST AV700083, representing almost the same sequence as AI668602, is specifically expressed in female breast tissue when using the 133 k chip.

Electronic northern provided evidence for expression in about 80% of both malignant and nonmalignant breast tissue specimens, the median level of expression increasing from 165 FU (relative fluorescence units) in normal tissue to 213 FU in cancer tissue. Noteworthy, a fraction of the breast cancer tissues demonstrated expression several factors above this level. Background expression in all available normal tissues was almost undetectable. Experimental evidence for breast-specific expression was provided by quantitative reverse transcription PCR using a multiple housekeeping gene standardized cDNA panel representing 16 normal tissues (BD Clontech) and CYP4Z1 cDNA specific primers. Expression was highest in breast (21.6 arbitrary units), followed by prostate (1.7 a.u.), testis (1.6 a.u.), lung (1.4 au), and liver (1 a.u.).

Further experimental evidence was provided by hybridizing a radioactive probe representing CYP4Z1 cDNA to a cancer profiling array (BD Clontech), representing cDNAs of more than 200 samples from paired normal/tumor tissue samples originating from 12 different organs. Remarkably, almost all of the breast cancer tissue samples showed expression, including three metastases originating from breast cancer.

Using a combination of data-mining (custom EST clustering) and 5′-RACE and 3′-RACE, the AI668620/AV700083 sequence was extended to full length cDNA, coding for a protein with homology to cytochrome P450 oxidases (CYP). From sequence of mouse and rat CYP, different researchers in the field of CYP had assumed that a yet unidentified human homolog had to exist. The CYP we identified is homologous with mouse and rat CYP, and we therefore conclude that it is indeed the predicted CYP4Z1.

The sequences disclosed herein incorporate the polynucleotide transcript of SEQ ID NO: 193 and the derived amino acid sequence (SEQ ID NO: 194) from said transcripts are available as targets for chemotherapeutic agents, especially anti-cancer agents, including antibodies specific for said polypeptides. The transcript for this gene is up in breast cancer and encodes a cytochrome P450 oxidase.

The cancer-related polynucleotide sequences disclosed herein correspond to gene sequences whose expression is indicative of the cancerous status of a given cell. Such sequences are substantially identical to SEQ ID NO: 193, which represents a transcript identified from the GenBank EST database and which exhibits cancer-specific expression. The polynucleotides of the invention are those that correspond to the sequence of SEQ ID NO: 193.

Other gene sequences whose expression is indicative of the cancerous status of a given cell are substantially identical to SEQ ID NO: 185, 186, 187 and 188, which represent different transcripts identified from the GenBank EST database and which exhibit cancer-specific expression. The polynucleotides of the invention are those that correspond to a sequence of SEQ ID NO: 185, 186, 187 and 188. Such sequences have been searched within the GenBank database, especially the EST database, with the following results:

| Type: | cell-surface tumor antigen |
|---|---|
| | therapeutic antibody target |
| Tissue: | breast and endometrium |
| AffyFragment-ID(s): | 125026, 142673 |
| | hypothetical protein FLJ22418 |
| Accession(s): | AA075632, AI799522 |
| Unigene cluster-ID(s): | Hs.36563 |
| Chromosomal location: | 1 |

Putative encoded protein sequences are shown as SEQ ID NO: 189 and 190.

Other different transcripts include sequences substantially identical to SEQ ID NO: 146-152, 159-165 and 171-177 with characteristics:

| Type: | cell-surface tumor antigen |
|---|---|
| | therapeutic antibody target |
| Tissue: | kidney |
| Accession(s): | AI479935, AI479935, AI186520 |
| Unigene cluster-ID(s): | Hs.61384 |
| Chromosomal location: | 3 |

Corresponding polypeptide sequences are shown as SEQ ID NO: 8-13, 21-26 and 34-39.

Other different transcripts include sequences substantially identical to SEQ ID NO: 140, 141, 142 and 143, with characteristics:

| Type: | cell-surface tumor antigen |
|---|---|
| | therapeutic antibody target |
| Tissue: | breast cancer, |
| | soft tissue fibromatosis, Wilms tumor |
| AffyFragment-ID(s): | 124040, 143095, 156021 |
| | KIAA1497 protein |
| Accession(s): | AI885003, AI857396, AI015776 |
| Unigene cluster-ID(s): | Hs.126085 |
| Chromosomal location: | 3 |

Other different transcripts include sequences substantially identical to SEQ ID NO: 129, 130, 131, 132 and 133, with characteristics:

| Type: | cell-surface tumor antigen |
|---|---|
| | therapeutic antibody target |
| Tissue: | lymphoma |
| AffyFragment-ID(s): | 140694, 144762 |
| | Homo sapiens SH2 domain-containing |
| | phosphatase anchor protein 1a (SPAP1) |
| | mRNA, complete cds, alternatively spliced |
| Accession(s): | AI630866, AA814007 |
| Unigene cluster-ID(s): | Hs.194976 |
| Chromosomal location: | 1 |

Other different transcripts include sequences substantially identical to SEQ ID NO: 138, with characteristics:

| Type: | cell-surface tumor antigen |
|---|---|
| | therapeutic antibody target |
| Tissue: | breast |
| AffyFragment-ID(s): | 149502 |
| Accession(s): | AI572043 |
| Unigene cluster-ID(s): | Hs.179082 |
| | EST |
| Chromosomal location: | 8 |

The genes disclosed herein may be used in any of the methods of the invention and modulation, as used herein, may include modulation of the gene, such as an increase or decrease in transcription or translation, and may include differences in the amount and/or the rate of production of RNA and/or polypeptide.

Fragments of such polynucleotides and polypeptides as are disclosed herein may also be useful in practicing the processes of the present invention. For example, a fragment, derivative or analog of a polypeptide encoded by one of the genes disclosed herein for use in the present invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such fragments, derivatives and analogs are deemed to be within the scope of those skilled in the art from the teachings herein.

The genes and gene products useful in practicing the methods of the present invention may likewise be obtained in an isolated or purified form. In addition, the polypeptide disclosed herein as being useful in practicing the processes of the invention include different types of proteins in terms of function so that some are enzymes, some are transcription factors and other may be cell surface receptors. Precisely how such cancer-linked proteins are used in the processes of the invention may thus differ depending on the function and cellular location of the protein and therefore modification, or optimization, of the methods disclosed herein may be desirable in light of said differences. For example, a cell-surface receptor is an excellent target for cytotoxic antibodies whereas a transcription factor or enzyme is a useful target for a small organic compound with anti-neoplastic activity. Expression products of the genes disclosed herein may also be in an isolated form.

Methods of producing vectors comprising genes disclosed herein, or recombinant cells expressing such genes, are well known to those skilled in the molecular biology art.

In one aspect, the present invention relates to a method for identifying a cancer-target gene, comprising:

a) identifying a gene that is at least 5 fold over-expressed in a cancer cell line and that maps to a chromosomal region with a CGH ratio of at least 1.25;

b) determining an RNA expression level of said gene of at least 1.5 fold in a tumor tissue compared to corresponding normal tissue in a genetic database,

c. determining that said gene encodes a protein domain known to be modulated, or shown to be modulated, by chemical compounds

wherein a gene that meets the criteria of a, b and c is considered to be a cancer-target gene,

thereby identifying a cancer-target gene.

The present invention also relates to a set of cancer-target genes identified using such methods. The genes disclosed herein form such a set. In addition, subsets of such sets are specifically contemplated by the invention. In a preferred embodiment, said gene was first identified as a cancer target gene using one or more of the methods of the invention.

In one non-limiting example, the gene is a gene selected from the group consisting of KIAA1274, NEK6, PAK2, PAK-4, STK38L, ACP1, ARHC, CDC6, CDK7, CDKN3, CRK7, DUSP16, FIGNL1, GUK1, ITPR2, KCNK1, KCNK5, PRO2000, RFC2 and RIPK2. These 20 genes are contained in Table 6 where they are described in terms of a consensus sequence along with identified polynucleotide transcripts and polypeptides.

In the methods of the invention, expression may be determined by determining transcription (to form RNA), as by measuring the rate or amount of RNA formed, or translation (to form protein), such as where antibodies may be used to determine the amount of polypeptide or protein formed from the gene in question or where the activity of such protein is determined, such as where the protein is an enzyme and the amount of enzyme activity can be determined.

In one preferred embodiment, the cell is a cancer cell and the determined difference in expression is a decrease in expression. In another embodiment, the cell is a recombinant cell, such as one comprising a gene as disclosed herein, and the difference in expression is a decrease in expression.

The present invention also relates to a method for identifying an anti-neoplastic agent comprising contacting a cell exhibiting neoplastic activity with a compound first identified as a cancer target gene modulator using one of the methods of the invention and detecting a decrease in said neoplastic activity after said contacting compared to when said contacting does not occur. In preferred embodiments, the neoplastic activity is accelerated cellular replication. In another preferred embodiment, the decrease in neoplastic activity results from the death of the cell. In a preferred embodiment, the compound is one that modulates, preferably inhibits, a gene disclosed herein.

The present invention also relates to a method for identifying an anti-neoplastic agent comprising administering to an animal exhibiting a cancerous condition an effective amount of a cancer target gene modulating agent by a method of the invention and detecting a decrease in said cancerous condition. In a preferred embodiment, the compound is one that modulates.

In accordance with the present invention, model cellular systems using cell lines, primary cells, or tissue samples are maintained in growth medium and may be treated with compounds that may be at a single concentration or at a range of concentrations. At specific times after treatment, cellular RNAs are isolated from the treated cells, primary cells or tumors, which RNAs are indicative of expression of selected genes. The cellular RNA is then divided and subjected to analysis that detects the presence and/or quantity of specific RNA transcripts, which transcripts may then be amplified for detection purposes using standard methodologies, such as, for example, reverse transcriptase polymerase chain reaction (RT-PCR), etc. The presence or absence, or levels, of specific RNA transcripts are determined from these measurements and a metric derived for the type and degree of response of the sample to the treated compound compared to control samples.

The methods of the invention may be used with a variety of cell lines or with primary samples from tumors maintained in vitro under suitable culture conditions for varying periods of time, or in situ in suitable animal models.

The nucleotides and polypeptides, as gene products, used in the processes of the present invention may comprise a recombinant polynucleotide or polypeptide, a natural polynucleotide or polypeptide, or a synthetic polynucleotide or polypeptide, or a recombinant polynucleotide or polypeptide.

Fragments of such polynucleotides and polypeptides as are disclosed herein may also be useful in practicing the processes of the present invention. For example, a fragment, derivative or analog of the polypeptide (SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide (such as a histidine hexapeptide) or a proprotein sequence. Such fragments, derivatives and analogs are deemed to be within the scope of those skilled in the art from the teachings herein.

In another aspect, the present invention relates to an isolated polypeptide, including a purified polypeptide, comprising an amino acid sequence at least 90% identical to the amino acid sequence of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769. In preferred embodiments, said isolated polypeptide comprises an amino acid sequence having sequence identity of at least 95%, preferably at least about 98%, and especially is identical to, the sequence of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769. The present invention also includes isolated active fragments of such polypeptides where said fragments retain the biological activity of the polypeptide or where such active fragments are useful as specific targets for cancer treatment, prevention or diagnosis. Thus, the present invention relates to any polypeptides, or fragments thereof, with sufficient sequence homology to the sequences disclosed herein as to be useful in the production of antibodies that react with (i.e., are selective or specific for) the polypeptides of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769 so as to be useful in targeting cells that exhibit such polypeptides, or fragments, on their surfaces, thereby providing targets for such antibodies and therapeutic agents associated with such antibodies.

The polynucleotides and polypeptides useful in practicing the processes of the present invention may likewise be obtained in an isolated or purified form. In addition, the polypeptide disclosed herein as being useful in practicing the processes of the invention are believed to be surface proteins present on cells, such as cancerous cells. Precisely how such cancer-linked proteins are used in the processes of the invention may thus differ depending on the therapeutic approach used. For example, cell-surface proteins, such as receptors, are desirable targets for toxic antibodies that can be generated against the polypeptides disclosed herein.

The sequence information disclosed herein, as derived from the GenBank submissions, can readily be utilized by those skilled in the art to prepare the corresponding full-length polypeptide by peptide synthesis. The same is true for either the polynucleotides or polypeptides disclosed herein for use in the methods of the invention.

The present invention relates to an isolated polypeptide, encoded by one of the polynucleotide transcripts disclosed herein, comprising an amino acid sequence homologous to an amino acid sequence selected from the group consisting of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769, wherein any difference between amino acid sequence in the isolated polypeptide and the sequence of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769 is due solely to conservative amino acid substitutions and wherein said isolated polypeptide comprises at least one immunogenic fragment. In a preferred embodiment, the present invention encompasses an isolated polypeptide comprising an amino acid sequence selected from the group consisting of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

Computer algorithms, for example SYFPEITHI (Rammensee H, et al., SYFPEITHI: database for MHC ligands and peptide motifs. Immunogenetics 50:213-219 (1999) can be used for the prediction of immunogenic HLA binding peptides like those disclosed herein and that can be subsequently identified by experimental methods well known in the art (Schmitz M, et al., EP.Generation of survivin-specific CD8+ T effector cells by dendritic cells pulsed with protein or selected peptides. Cancer Research, 60(17):4845-9 (2000); Kiessling A, et al., EP.Prostate stem cell antigen: Identification of immunogenic peptides and assessment of reactive CD8+ T cells in prostate cancer patients, Int'l J. Cancer, 102(4):390-7 (2002).

Methods of producing recombinant cells and vectors useful in preparing the polynucleotides and polypeptides disclosed herein are well known to those skilled in the molecular biology art. See, for example, Sambrook, et al., Molecular Cloning: A Laboratory Manual, Second Edition, Cold Spring Harbor, N.Y., (1989), Wu et al., Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

In one aspect, the present invention relates to a process for identifying an agent that modulates the activity of a cancer-related gene comprising:

(a) contacting a compound with a cell containing a gene that corresponds to a polynucleotide having a sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768 and under conditions promoting the expression of said gene; and

(b) detecting a difference in expression of said gene relative to when said compound is not present

thereby identifying an agent that modulates the activity of a cancer-related gene.

In specific embodiments of such, said polynucleotide sequence may be the same as one of said sequences or may be the complement of any of the sequences disclosed herein.

In another aspect, the present invention relates to a process for identifying an anti-neoplastic agent comprising contacting a cell exhibiting neoplastic activity with a compound first identified as a cancer related gene modulator using an assay process disclosed herein and detecting a decrease in said neoplastic activity after said contacting compared to when said contacting does not occur. Such neoplastic activity may include accelerated cellular replication and/or metastasis, and the decrease in neoplastic activity preferably results from the death of the cell.

The present invention also relates to a process for identifying an anti-neoplastic agent comprising administering to an animal exhibiting a cancer condition an effective amount of an agent first identified according to a process of one of one of the assays disclosed according to the invention and detecting a decrease in said cancerous condition.

In accordance with the present invention, such assays rely on methods of determining the activity of the gene in question. Such assays are advantageously based on model cellular systems using cancer cell lines, primary cancer cells, or cancerous tissue samples that are maintained in growth medium and treated with compounds at a single concentration or at a range of concentrations. At specific times after treatment, cellular RNAs are conveniently isolated from the treated cells or tissues, which RNAs are indicative of expression of selected genes. The cellular RNA is then divided and subjected to differential analysis that detects the presence and/or quantity of specific RNA transcripts, which transcripts may then be amplified for detection purposes using standard methodologies, such as, for example, reverse transcriptase polymerase chain reaction (RT-PCR), etc. The presence or absence, or concentration levels, of specific RNA transcripts are determined from these measurements. The polynucleotide sequences disclosed herein are readily used as probes for the detection of such RNA transcripts and thus the measurement of gene activity and expression.

The polynucleotides of the invention can include fully operational genes with attendant control or regulatory sequences or merely a polynucleotide sequence encoding the corresponding polypeptide or an active fragment or analog thereof.

Expression of the polynucleotide sequences disclosed herein are specific to types of cancer and may be specific for the cancerous state, useful gene modulation is downward modulation, so that, as a result of exposure to an antineoplastic agent identified by the screening assays herein, the corresponding gene of the cancerous cell is expressed at a lower level (or not expressed at all) when exposed to the agent as compared to the expression when not exposed to the agent. Where said chemical agent causes a gene of the tested cell to be expressed at a lower level than the same genes of the reference, this is indicative of downward modulation and indicates that the chemical agent to be tested has anti-neoplastic activity.

In carrying out the assays disclosed herein, relative antineoplastic activity may be ascertained by the extent to which a given chemical agent modulates the expression of genes present in a cancerous cell. Thus, a first chemical agent that modulates the expression of a gene associated with the cancerous state (i.e., a gene corresponding to one or more of the polynucleotide transcripts disclosed herein) to a larger degree than a second chemical agent tested by the assays of the invention is thereby deemed to have higher, or more desirable, or more advantageous, anti-neoplastic activity than said second chemical agent.

The gene expression to be measured is commonly assayed using RNA expression as an indicator. Thus, the greater the level of RNA (for example, messenger RNA or mRNA) detected the higher the level of expression of the corresponding gene. Thus, gene expression, either absolute or relative, is determined by the relative expression of the RNAs encoded by such genes.

RNA may be isolated from samples in a variety of ways, including lysis and denaturation with a phenolic solution containing a chaotropic agent (e.g., trizol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies.

Normally, prior to applying the processes of the invention, steady state RNA expression levels for the genes, and sets of genes, disclosed herein will have been obtained. It is the steady state level of such expression that is affected by potential anti-neoplastic agents as determined herein. Such steady state levels of expression are easily determined by any methods that are sensitive, specific and accurate. Such methods include, but are in no way limited to, real time quantitative polymerase chain reaction (PCR), for example, using a Perkin-Elmer 7700 sequence detection system with gene specific primer probe combinations as designed using any of several commercially available software packages, such as Primer Express software, solid support based hybridization array technology using appropriate internal controls for quantitation, including filter, bead, or microchip based arrays, solid support based hybridization arrays using, for example, chemiluminescent, fluorescent, or electrochemical reaction based detection systems.

The gene expression indicative of a cancerous state need not be characteristic of every cell of a given tissue. Thus, the methods disclosed herein are useful for detecting the presence of a cancerous condition within a tissue where less than all cells exhibit the complete pattern. Thus, by non-limiting example, a selected gene corresponding to the sequence of SEQ ID NO: 1, may be found, using appropriate probes, either DNA or RNA, to be present in as little as 60% of cells derived from a sample of tumorous, or malignant, tissue. In a highly preferred embodiment, such gene pattern is found to be present in at least 100% of cells drawn from a cancerous tissue and absent from at least 100% of a corresponding normal, non-cancerous, tissue sample.

Expression of a gene may be related to copy number, and changes in expression may be measured by determining copy number. Such change in gene copy number may be determined by determining a change in expression of messenger RNA encoded by a particular gene sequence, especially that of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768. Also in accordance with the present invention, said gene may be a cancer initiating or facilitating gene. In carrying out the methods of the present invention, a cancer facilitating gene is a gene that, while not directly initiating tumor formation or growth, acts, such as through the actions of its expression product, to direct, enhance, or otherwise facilitate the progress of the cancerous condition, including where such gene acts against genes, or gene expression products, that would otherwise have the effect of decreasing tumor formation and/or growth.

Although the expression of a gene corresponding to one of said sequences may be indicative of a cancerous status for a given cell, the mere presence of such a gene may not alone be sufficient to achieve a malignant condition and thus the level of expression of such gene may also be a significant factor in determining the attainment of a cancerous state. Thus, it becomes essential to also determine the level of expression of a gene as disclosed herein, including substantially similar sequences, as a separate means of diagnosing the presence of a cancerous status for a given cell, groups of cells, or tissues, either in culture or in situ.

The level of expression of the polypeptides disclosed herein is also a measure of gene expression, such as polypeptides having sequence identical, or similar to, any polypeptide encoded by a sequence of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768, especially a polypeptide whose amino acid sequence is the sequence of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

In accordance with the foregoing, the present invention specifically contemplates a method for determining the cancerous status of a cell to be tested, comprising determining the level of expression in said cell of a gene that includes one of the nucleotide sequences disclosed herein including sequences substantially identical to said sequences, or characteristic fragments thereof, or the complements of any of the foregoing and then comparing said expression to that of a cell known to be non-cancerous whereby the difference in said expression indicates that said cell to be tested is cancerous.

In accordance with the invention, gene expression for a gene that includes as a portion of one of the sequences of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768, or a complement of one of these, is preferably determined by use of a probe that is a fragment of such nucleotide sequence, it is to be understood that the probe may be formed from a different portion of the gene. Expression of the gene may also be determined by use of a nucleotide probe that hybridizes to messenger RNA (mRNA) transcribed from a portion of the gene other than the specific nucleotide sequence disclosed herein.

It should be noted that some genes may be oncogenes and encode proteins that are directly involved in the cancerous process and thereby promote the occurrence of cancer in an animal. In addition, other genes may serve to suppress the cancerous state in a given cell or cell type and thereby work against a cancerous condition forming in an animal. Other genes may simply be involved either directly or indirectly in the cancerous process or condition and may serve in an ancillary capacity with respect to the cancerous state. All such types of genes are deemed with those to be determined in accordance with the invention as disclosed herein. Thus, the gene determined by said process of the invention may be an oncogene, or the gene determined by said process may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. In addition, the gene determined by said process may be a cancer suppressor gene, which gene works either directly or indirectly to suppress the initiation or progress of a cancerous condition. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

Polynucleotides encoding the same proteins as any of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768, including complements thereof, regardless of the percent identity of such sequences, are also specifically contemplated by any of the methods of the present invention. Thus, any such sequences are available for use in carrying out any of the methods disclosed according to the invention. Such sequences also include any open reading frames present within any of these sequences.

A gene disclosed according to the invention also encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a polynucleotide having a sequence as disclosed herein.

The sequences disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence, or they may be wholly synthetic in origin for purposes of practicing the processes of the invention. Because of the processing that may take place in transforming the initial RNA transcript into the final mRNA, the sequences disclosed herein may represent less than the full genomic sequence. They may also represent sequences derived from ribosomal and transfer RNAs. Consequently, the gene as present in the cell (and representing the genomic sequence) and the polynucleotide transcripts disclosed herein, including cDNA sequences, may be identical or may be such that the cDNAs contain less than the full genomic sequence. Such genes and cDNA sequences are still considered “corresponding sequences” (as defined elsewhere herein) because they both encode the same or related RNA sequences (i.e., related in the sense of being splice variants or RNAs at different stages of processing). Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous cells and are used to determine gene activity or expression because they represent the same sequence or are complementary to RNAs encoded by the gene. Such a gene also includes different alleles and splice variants that may occur in the cells used in the methods of the invention, such as where recombinant cells are used to assay for anti-neoplastic agents and such cells have been engineered to express a polynucleotide as disclosed herein, including cells that have been engineered to express such polynucleotides at a higher level than is found in non-engineered cancerous cells or where such recombinant cells express such polynucleotides only after having been engineered to do so. Such engineering includes genetic engineering, such as where one or more of the polynucleotides disclosed herein has been inserted into the genome of such cell or is present in a vector.

Such cells, especially mammalian cells, may also be engineered to express on their surfaces one or more of the polypeptides of the invention for testing with antibodies or other agents capable of masking such polypeptides and thereby removing the cancerous nature of the cell. Such engineering includes both genetic engineering, where the genetic complement of the cells is engineered to express the polypeptide, as well as non-genetic engineering, whereby the cell has been physically manipulated to incorporate a polypeptide of the invention in its plasma membrane, such as by direct insertion using chemical and/or other agents to achieve this result.

In accordance with the foregoing, the present invention includes anti-cancer agents that are themselves either polypeptides, or small chemical entities, that affect the cancerous process, including initiation, suppression or facilitation of tumor growth, either in vivo or ex vivo. Said cancer modulating agent will have the effect of decreasing gene expression.

The present invention thus also relates to a method for reducing cancer cell peoliferation, or protecting against cancer, comprising contacting a cancerous cell with an agent having activity against an expression product encoded by a gene or polynucleotide sequence as disclosed herein. The present invention also relates to a process for treating cancer comprising contacting a cancerous cell with an agent having activity against an expression product encoded by a gene or polynucleotide sequence as disclosed herein. In one such embodiment, the cancerous cell is contacted in vivo. In another example, the agent has affinity for said expression product.

In a preferred embodiment, such agent is an antibody that is specific or selective for a polypeptide comprising an amino acid sequence selected from SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

The present invention is also directed to such uses of the compositions of polypeptides and antibodies disclosed herein. Such uses include a process for treating cancer in an animal afflicted therewith comprising administering to said animal an amount of an immunogenic composition of one or more of the polypeptides disclosed herein where such amount if an amount sufficient to elicit the production of cytotoxic T lymphocytes specific for a polypeptide incorporating a sequence of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769. In a preferred embodiment, the animal to be so treated is a human patient.

The proteins encoded by the genes disclosed herein due to their expression, or elevated expression, in cancer cells, represent highly useful therapeutic targets for “targeted therapies” utilizing such affinity structures as, for example, antibodies coupled to some cytotoxic agent. In such methodology, it is advantageous that nothing need be known about the endogenous ligands or binding partners for such cell surface molecules. Rather, an antibody or equivalent molecule that can specifically recognize the cell surface molecule (which could include an artificial peptide, a surrogate ligand, and the like) that is coupled to some agent that can induce cell death or a block in cell cycling offers therapeutic promise against these proteins. Thus, such approaches include the use of so-called suicide “bullets” against intracellular proteins. For example, monoclonal antibodies may readily by produced by methods well known in the art, for example, the method of Kohler and Milstein (see: Nature, 256:495 (1975).

With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, such as cows, goats and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. Canonical CDR regions have been described by, for example, Kabat et al., J. Biol. Chem. 252:6609-6616 (1977).

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors.

In one aspect, the present invention relates to immunoglobulins, or antibodies, as described herein, that are specific for polypeptides having an amino acid sequence of one of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769. Such antibodies may commonly be in the form of a composition, especially a pharmaceutical composition.

Such antibodies, by themselves, may have therapeutic value in that they are able to bind to, and thereby tie up, surface sites on cancerous cells. Where such sites have some type of function to perform (i.e., where they are surface enzymes, or channel structures, or structures that otherwise facilitate, actively or passively, the transport of nutrients and other vital materials to the cell). Molecules that bind to such sites and thereby interfere with such activities can prove to have a therapeutic effect in that the result of such binding is to remove sources of nutrients from such cells, thereby interfering with growth and replication. In like manner, such binding may serve to remove vital enzyme activities from the cell's functional repertoire, thereby also interfering with viability and/or the ability of the cell to multiply or metastasize. In addition, by binding to such surface sites, the antibodies may serve to prevent the cells from reacting to environmental agents, such as cytokines and the like, that may facilitate growth, replication and metastasis, thereby further reducing the cancerous status of such cell and ameliorating the cancerous condition in a patient, even without proving fatal to the cell or cells so affected.

An antibody may be polyclonal, monoclonal, recombinant or synthetic in origin. In one such embodiment, said antibody is associated, either covalently or non-covalently, with a cytotoxic agent, for example, an apoptotic agent. It is thus contemplated that the antibody acts a targeted vector for guiding an associated therapeutic agent to a cancerous cell, such as a cell expressing a polypeptide homologous to, if not identical to, a polypeptide as disclosed herein.

Where the cytotoxic agent is itself a polypeptide, said may be linked directly to an antibody specific for a surface target on a cancer cell, such as where the polypeptide represents an extension of the amino acid chain of the antibody. In alternative embodiments, such molecules may be covalently linked through a linker sequence of long or short duration, such as an amino acid sequence of 5 to 10 residues in length. Where the cytotoxic agents is some small organic molecule, such as a small organic compound, or some type of apoptotic agent, this may be covalently bonded to the antibody molecule or may be attached by some other type of non-covalent linkage, including hydrophobic and electrostatic linkages. Methods for forming such linkages, especially covalent linkages, are well known to those skilled in the art.

The antibodies disclosed herein may also serve as targeting vectors for much larger structures, such as liposomes. In one such embodiment, an antibody is part of, or otherwise linked to, or associated with, a membranous structure, preferably a liposome or possibly some type of cellular organelle, which acts as a reservoir for a cytotoxic agent, such as ricin. The antibody then acts to target said liposome to a cancerous tissue in an animal, whereupon the liposome provides a source of cytotoxic agents for localized treatment of a solid tumor or other type of neoplasm.

The present invention further encompasses an immunogenic composition comprising a polypeptide disclosed herein, as well as compositions formed using antibodies specific for these polypeptides.

Methods well known in the art for making formulations are found in, for example, Remington: The Science and Practice of Pharmacy, (19th ed.) Ed. A.R. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel. It should be noted that, where the therapeutic agent to be administered is an immunoconjugate, these sometimes contain chemical linkages that are somewhat labile in aqueous media and therefor must be stored prior to administration is a more stable environment, such as in the form of a lyophilized powder.

Such an agent can be a single molecular structure, comprising both affinity portion and anti-cancer activity portions, wherein said portions are derived from separate molecules, or molecular structures, possessing such activity when separated and wherein such agent has been formed by combining said portions into one larger molecular structure, such as where said portions are combined into the form of an adduct. Said anti-cancer and affinity portions may be joined covalently, such as in the form of a single polypeptide, or polypeptide-like, structure or may be joined non-covalently, such as by hydrophobic or electrostatic interactions, such structures having been formed by means well known in the chemical arts. Alternatively, the anti-cancer and affinity portions may be formed from separate domains of a single molecule that exhibits, as part of the same chemical structure, more than one activity wherein one of the activities is against cancer cells, or tumor formation or growth, and the other activity is affinity for an expression product produced by expression of genes related to the cancerous process or condition.

In one embodiment of the present invention, a chemical agent, such as a protein or other polypeptide, is joined to an agent, such as an antibody, having affinity for an expression product of a cancerous cell, such as a polypeptide or protein encoded by a gene related to the cancerous process, preferably a gene as disclosed herein according to the present invention, most preferably a polypeptide sequence disclosed herein. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

Other genes within the cancer cell that are regulated in a manner similar to that of the genes disclosed herein and thus change their expression in a coordinated way in response to chemical compounds represent genes that are located within a common metabolic, signaling, physiological, or functional pathway so that by analyzing and identifying such commonly regulated groups of genes (groups that include the gene, or similar sequences, disclosed according to the invention, one can (a) assign known genes and novel genes to specific pathways and (b) identify specific functions and functional roles for novel genes that are grouped into pathways with genes for which their functions are already characterized or described. For example, one might identify a group of 10 genes, at least one of which is the gene as disclosed herein, that change expression in a coordinated fashion and for which the function of one, such as the polypeptide encoded by the sequence disclosed herein, is known then the other genes are thereby implicated in a similar function or pathway and may thus play a role in the cancer-initiating or cancer-facilitating process. In the same way, if a gene were found in normal cells but not in cancer cells, or happens to be expressed at a higher level in normal as opposed to cancer cells, then a similar conclusion may be drawn as to its involvement in cancer, or other diseases. Therefore, the processes disclosed according to the present invention at once provide a novel means of assigning function to genes, i.e. a novel method of functional genomics, and a means for identifying chemical compounds that have potential therapeutic effects on specific cellular pathways. Such chemical compounds may have therapeutic relevance to a variety of diseases outside of cancer as well, in cases where such diseases are known or are demonstrated to involve the specific cellular pathway that is affected.

The present invention specifically contemplates use of antibodies against the polypeptides encoded by the polynucleotides corresponding to the genes disclosed herein, whereby said antibodies are conjugated to one or more cytotoxic agents so that the antibodies serve to target the conjugated immunotoxins to a region of cancerous activity, such as a solid tumor. For many known cytotoxic agents, lack of selectivity has presented a drawback to their use as therapeutic agents in the treatment of malignancies. For example, the class of two-chain toxins, consisting of a binding subunit (or B-chain) linked to a toxic subunit (A-chain) are extremely cytotoxic. Thus, such agents as ricin, a protein isolated from castor beans, kills cells at very low concentrations (even less than 10⁻¹¹ M) by inactivating ribosomes in said cells (see, for example, Lord et al., Ricin: structure, mode of action, and some current applications. Faseb J, 8: 201-208 (1994), and Blättler et al., Realizing the full potential of immunotoxins. Cancer Cells, 1: 50-55 (1989)). While isolated A-chains of protein toxins that functionally resemble ricin A-chain are only weakly cytotoxic for intact cells (in the concentration range of 10⁻⁷ to 10⁻⁶ M), they are very potent cytotoxic agents inside the cells. Thus, a single molecule of the A-subunit of diphtheria toxin can kill a cell once inside (see: Yamaizumi et al., One molecule of diphtheria toxin fragment A introduced into a cell can kill the cell. Cell, 15: 245-250, 1978).

The present invention solves this selectivity problem by using antibodies specific for antigens present on cancer cells to target the cytotoxins to said cells. In addition, use of antibodies decreases toxicity because the antibodies are non-toxic until they reach the tumor and, because the cytotoxin is bound to the antibody, it is presented with less opportunity to cause damage to non-targeted tissues.

In addition, use of such antibodies alone can provide therapeutic effects on the tumor through the antibody-dependent cellular cytotoxic response (ADCC) and complement-mediated cell lysis mechanisms.

A number of recombinant immunotoxins (for example, consisting of Fv regions of cancer specific antibodies fused to truncated bacterial toxins) are well known (see, for example, Smyth et al., Specific targeting of chlorambucil to tumors with the use of monoclonal antibodies, J. Natl. Cancer Inst., 76(3):503-510 (1986); Cho et al., Single-chain Fv/folate conjugates mediate efficient lysis of folate-receptor-positive tumor cells, Bioconjug. Chem., 8(3):338-346 (1997)). As noted in the literature, these may contain, for example, a truncated version of Pseudomonas exotoxin as a toxic moiety but the toxin is modified in such a manner that by itself it does not bind to normal human cells, but it retains all other functions of cytotoxicity. Here, recombinant antibody fragments target the modified toxin to cancer cells which are killed, such as by direct inhibition of protein synthesis, or by concomitant induction of apoptosis. Cells that are not recognized by the antibody fragment, because they do not carry the cancer antigen, are not affected. Good activity and specificity has been observed for many recombinant immunotoxins in in vitro assays using cultured cancer cells as well as in animal tumor models. Ongoing clinical trials provide examples where the promising pre-clinical data correlate with successful results in experimental cancer therapy. (see, for example, Brinkmann U., Recombinant antibody fragments and immunotoxin fusions for cancer therapy, In Vivo (2000) 14:21-27).

While the safety of employing immunoconjugates in humans has been established, in vivo therapeutic results have been less impressive. Because clinical use of mouse MAbs in humans is limited by the development of a foreign anti-globulin immune response by the human host, genetically engineered chimeric human-mouse MAbs have been developed by replacing the mouse Fc region with the human constant region. In other cases, the mouse antibodies have been “humanized” by replacing the framework regions of variable domains of rodent antibodies by their human equivalents. Such humanized and engineered antibodies can even be structurally arranged to have specificities and effector functions determined by design and which characteristics do not appear in nature. The development of bi-specific antibodies, having different binding ends so that more than one antigenic site can be bound, have proven useful in targeting cancer cells. Thus, such antibody specificity has been improved by chemical coupling to various agents such as bacterial or plant toxins, radionuclides or cytotoxic drugs and other agents. (see, for example, Bodey, B. et al). Genetically engineered monoclonal antibodies for direct anti-neoplastic treatment and cancer cell specific delivery of chemotherapeutic agents. Curr Pharm Des (2000) Feb;6(3):261-76). See also, Garnett, M. C., Targeted drug conjugates: principles and progress. Adv. Drug Deliv. Rev. (2001 Dec. 17) 53(2):171-216; Brinkmann et al., Recombinant immunotoxins for cancer therapy. Expert Opin Biol Ther. (2001) 1(4):693-702.

Among the cytotoxic agents specifically contemplated for use as immunoconjugates according to the present invention are Calicheamicin, a highly toxic enediyne antibiotic isolated from Micromonospora echinospora ssp. Calichensis, and which binds to the minor groove of DNA to induce double strand breaks and cell death (see: Lee et al., Calicheamicins, a novel family of antitumor antibiotics. 1. Chemistry and partial structure of calichemicin g₁. J Am Chem Soc, 109: 3464-3466 (1987); Zein et al., Calicheamicin gamma 1I: an antitumor antibiotic that cleaves double-stranded DNA site specifically, Science, 240: 1198-1201 (1988)). Useful derivatives of the calicheamicins include mylotarg and 138H11-Camθ. Mylotarg is an immunoconjugate of a humanized anti-CD33 antibody (CD33 being found in leukemic cells of most patients with acute myeloid leukemia) and N-acetyl gamma colicheamicin dimethyl hydrazide, the latter of which is readily coupled to an antibody of the present invention (in place of the anti-CD33 but which can also be humanized by substitution of human framework regions into the antibody during production as described elsewhere herein) to form an immunoconjugate of the invention. (see: Hamann et al. Gemtuzumab Ozogamicin, A Potent and Selective Anti-CD33 Antibody-Calicheamicin Conjugate for Treatment of Acute Myeloid Leukemia, Bioconjug. Chem. 13, 47-58 (2002)) For use with 138H11-Camθ, 138H11 is an anti-γ-glutamyl transferase antibody coupled to theta calicheamicin through a disulfide linkage and found useful in vitro against cultured renal cell carcinoma cells. (see: Knoll et al., Targeted therapy of experimental renal cell carcinoma with a novel conjugate of monoclonal antibody 138H11 and calicheamicin θ₁^{I}, Cancer Res, 60: 6089-6094 (2000) The same linkage may be utilized to link this cytotoxic agent to an antibody of the present invention, thereby forming a targeting structure for cancer cells.

Also useful in forming the immunoconjugates of the invention is DC1, a disulfide-containing analog of adozelesin, that kills cells by binding to the minor groove of DNA, followed by alkylation of adenine bases. Adozelesin is a structural analog of CC-1065, an anti-tumor antibiotic isolated from microbial fermentation of Streptomyces zelensis, and is about 1,000 fold more toxic to cultured cell lines that other DNA interacting agents, such as cis-platin and doxorubicin. This agent is readily linked to antibodies through the disulfide bond of adozelesin. (see: Chari et al., Enhancement of the selectivity and antitumor efficacy of a CC-1065 analogue through immunoconjugate formation, Cancer Res, 55: 4079-4084 (1995)).

Maytansine, a highly cytotoxic microtubular inhibitor isolated from the shrub Maytenus serrata found to have little value in human clinical trials, is much more effective in its derivatized form, denoted DM1, containing a disulfide bond to facilitate linkage to antibodies, is up to 10-fold more cytotoxic (see: Chari et al., Immunoconjugates containing novel maytansinoids: promising anticancer drugs, Cancer Res, 52: 127-131 (1992)). These same in vitro studies showed that up to four DM1 molecules could be linked to a single immunoglobulin without destroying the binding affinity. Such conjugates have been used against breast cancer antigens, such as the neu/HER2/erbB-2 antigen. (see: Goldmacher et al., Immunogen, Inc., (2002) in press); also see Liu, C. et al., Eradication of large colon tumor xenografts by targeted delivery of maytansinoids, Proc. Natl. Acad. Sci. USA, 93, 8618-8623 (1996)). For example, Liu et al. (1996) describes formation of an immunoconjugate of the maytansinoid cytotoxin DM1 and C242 antibody, a murine IgG1 immunoglobulin, available from Pharmacia and which has affinity for a mucin-like glycoprotein variably expressed by human colorectal cancers. The latter immunoconjugate was prepared according to Chari et al., Cancer Res., 52:127-131 (1992) and was found to be highly cytotoxic against cultured colon cancer cells as well as showing anti-tumor effects in vivo in mice bearing subcutaneous COLO 205 human colon tumor xenografts using doses well below the maximum tolerated dose.

In addition, there are a variety of protein toxins (cytotoxic proteins), which include a number of different classes, such as those that inhibit protein synthesis: ribosome-inactivating proteins of plant origin, such as ricin, abrin, gelonin, and a number of others, and bacterial toxins such as pseudomonas exotoxin and diphtheria toxin.

Another useful class is the one including taxol, taxotere, and taxoids. Specific examples include paclitaxel (taxol), its analog docetaxel (taxotere), and derivatives thereof. The first two are clinical drugs used in treating a number of tumors while the taxoids act to induce cell death by inhibiting the de-polymerization of tubulin. Such agents are readily linked to antibodies through disulfide bonds without disadvantageous effects on binding specificity.

In one instance, a truncated Pseudomonas exotoxin was fused to an anti-CD22 variable fragment and used successfully to treat patients with chemotherapy-resistant hairy-cell leukemia. (see: Kreitman et al., Efficacy of the anti-CD22 recombinant immunotoxin BL22 in chemotherapy-resistant hairy-cell leukemia, N Engl J Med, 345: 241-247 (2001)) Conversely, the cancer-linked peptides of the present invention offer the opportunity to prepare antibodies, recombinant or otherwise, against the appropriate antigens to target solid tumors, preferably those of malignancies of prostate tissue, breast tissue, and the like, using the same or similar cytotoxic conjugates. Thus, many of the previously used immunoconjugates have been formed using antibodies against general antigenic sites linked to cancers whereas the antibodies formed using the peptides disclosed herein are more specific and target the antibody-cytotoxic agent to a particular tissue or organ, thus further reducing toxicity and other undesirable side effects.

In addition, the immunoconjugates formed using the antibodies prepared against the cancer-linked antigens disclosed herein can be formed by any type of chemical coupling. Thus, the cytotoxic agent of choice, along with the immunoglobulin, can be coupled by any type of chemical linkage, covalent or non-covalent, including electrostatic linkage, to form the immunoconjugates of the present invention.

When used as immunoconjugates, the antitumor agents of the present invention represent a class of pro-drugs that are relatively non-toxic when first administered to an animal (due mostly to the stability of the immunoconjugate), such as a human patient, but which are targeted by the conjugated immunoglobulin to a cancer cell where they then exhibit good toxicity. The tumor-related, associated, or linked, antigens, preferably those presented herein, serve as targets for the antibodies (monoclonal, recombinant, and the like) specific for said antigens. The end result is the release of active cytotoxic agent inside the cell after binding of the immunoglobulin portion of the immunoconjugate.

The cited references describe a number of useful procedures for the chemical linkage of cytotoxic agents to immunoglobulins and the disclosures of all such references cited herein are hereby incorporated by reference in their entirety. For other reviews see Ghetie et al., Immunotoxins in the therapy of cancer: from bench to clinic, Pharmacol Ther, 63: 209-234 (1994), Pietersz et al. The use of monoclonal antibody immunoconjugates in cancer therapy, Adv Exp Med Biol, 353:169-179 (1994), and Pietersz, G. A. The linkage of cytotoxic drugs to monoclonal antibodies for the treatment of cancer, Bioconjug Chem, 1:89-95 (1990).

Thus, the present invention provides highly useful cancer-associated antigens for generation of antibodies for linkage to a number of different cytotoxic agents which are already known to have some in vitro toxicity and possess chemical groups available for linkage to antibodies.

The present invention also relates to a process that comprises a method for producing a product, including the generation of test data, comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

It should be cautioned that, in carrying out the procedures of the present invention as disclosed herein, whether to form immunoconjugates or screen for other antitumor agents using the genes and polypeptides disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art. The following example shows how a potential anti-neoplastic agent may be identified using one or more of the genes disclosed herein.

### EXAMPLE

Cells (e.g., SW480 cells, ovarian tumor cells, prostate tumor cells, breast cancer cells or other cancerous cells) are grown to a density of 10⁵ cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1×PCR buffer, RNAsin, reverse transcriptase, nucleoside triphosphates, amplitaq gold, tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perlin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference gene is then calculated and a relative expression value determined for all of the samples used. This procedure is then repeated for other genes functionally related to the gene as disclosed herein and the level of function, or expression, noted. The relative expression ratios for each pair of genes is determined (i.e., a ratio of expression is determined for each target gene versus each of the other genes for which expression is measured, where each gene's absolute expression is determined relative to the reference gene for each compound, or chemical agent, to be screened). The samples are then scored and ranked according to the degree of alteration of the expression profile in the treated samples relative to the control. The overall expression of the particular gene relative to the controls, as modulated by one chemical agent relative to another, is also ascertained. Chemical agents having the most effect on a given gene, or set of genes, are considered the most anti-neoplastic.

### REFERENCES

Walter A. Blättler and Ravi Chari: Drugs to enhance the therapeutic potency of anti-cancer antibodies: antibody-drug conjugates as tumor-activated prodrugs. In Anticancer Agents—Frontiers in Cancer Chemotherapy (Iwao Ojima, Gregory D. Vite, Karl-Heinz Altmann, Eds.), American Chemical Society, pp. 317-338(2001).
Dan L. Longo, Patricia L. Duffey, John G. Gribben, Elaine S. Jaffe, Brendan D. Curti, Barry L. Gause, John E. Janik, Virginia M. Braman, Dixie Esseltine, Wyndham H. Wilson, Dwight Kaufman, Robert E. Wittes, Lee M. Nadler, and Walter J. Urba: Combination chemotherapy followed by an Immunotoxin (Anti-B4-blocked Ricin) in patients with indolent lymphoma: results of a Phase II study. Cancer J. 6, 146-150 (2000).
Walter A. Blättler and John M. Lambert: Preclinical immunotoxin development. In Monoclonal Antibody-Based Therapy of Cancer (M. Grossbard, Ed.), Marcel Dekker, Inc. NY, N.Y., pp. 1-22 (1998).
Ravi V.J. Chari: Targeted delivery of chemotherapeutics: tumor-activated prodrug therapy. In Advanced Drug Delivery Reviews, Elsevier Science B.V., pp. 89-104 (1998).
David T. Scadden, David P. Schenkein, Zale Bernstein, Barry Luskey, John Doweiko, Anil Tulpule, and Alexandra M. Levine: Immunotoxin combined with chemotherapy for patients with AIDS-related Non-Hodgkin's Lymphoma. Cancer 83, 2580-2587 (1998).
Changnian Liu and Ravi VJ Chari: The development of antibody delivery systems to target cancer with highly potent maytansinoids. Exp. Opi. Invest Drugs 6, 169-172 (1997).
A. C. Goulet, Viktor S. Goldmacher, John M. Lambert, C. Baron, Dennis C. Roy and E. Kouassi: Conjugation of blocked ricin to an anti-CD19 monoclonal antibody increases antibody-induced cell calcium mobilization and CD19 internalization. Blood 90, 2364-2375 (1997).

Changnian Liu, John M. Lambert, Beverly A. Teicher, Walter A. Blättler, and Rosemary O'Connor: Cure of multidrug-resistant human B-cell lymphoma xenografts by combinations of anti-B4-blocked ricin and chemotherapeutic drugs. Blood 87, 3892-3898 (1996).
Rajeeva Singh, Lana Kats, Walter A. Blättler, and John M. Lambert: Formation of N-Substituted 2-Iminothiolanes when amino groups in proteins and peptides are modified by 2-Iminothiolane. Anal. Biochem. 236, 114-125 (1996).
Changnian Liu, B. Mitra Tadayoni, Lizabeth A. Bourret, Kristin M. Mattocks, Susan M. Derr, Wayne C. Widdison, Nancy L. Kedersha, Pamela D. Ariniello, Victor S. Goldmacher, John M. Lambert, Walter A. Blättler, and Ravi V.J. Chari: Eradication of large colon tumor xenografts by targeted delivery of maytansinoids. Proc. Natl. Acad. Sci. USA 93, 8618-8623 (1996).
Denis C. Roy, Sophie Ouellet, Christiane Le Houiller, Pamela D. Ariniello, Claude Perreault and John M. Lambert: Elimination of neuroblastoma and small-cell lung cancer cells with an anti-neural cell adhesion molecule immunotoxin. J. Natl. Cancer Inst. 88, 1136-1145 (1996).

Walter A. Blättler, Ravi V.J. Chari and John M. Lambert: Immunoconjugates. In Cancer Therapeutics: Experimental and Clinical Agents. (B. Teicher, Ed.), Humana Press, Totowa, N.J., pp. 371-394 (1996).
Michael L Grossbard, John M. Lambert, Victor S. Goldmacher, Arnold S. Freedman, Jeanne Kinsella, Danny P. Ducello, Susan N. Rabinowe, Laura Elisea, Felice Carol, James A. Taylor, Walter A. Blättler, Carol L. Epstein, and Lee M. Nadler: Anti-B4-blocked Ricin: A phase I trial of 7 day continuous infusion in patients with B-cell neoplasms. J. Clin. Oncol. 11, 726-737 (1993).
Michael L. Grossbard, John G. Gribben, Arnold S. Freedman, John M. Lambert, Jeanne Kinsella, Susan N. Rabinowe, Laura Eliseo, James A. Taylor, Walter A. Blättler, Carol L. Epstein, and Lee M. Nadler: Adjuvant immunotoxin therapy with anti-B4-blocked ricin following autologous bone marrow transplantation for patients with B-cell Non-Hodgkin's lymphoma. Blood 81, 2263-2271 (1993).
Sudhir A. Shah, Patricia M. Halloran, Cynthia A. Ferris, Beth A. Levine, Lizabeth A. Bourret, Victor S. Goldmacher, and Walter A. Blättler: Anti-B4-blocked Ricin immunotoxin shows therapeutic efficacy in four different SCID mouse tumor models. Cancer Res. 53, 1360-1367 (1993).
Ravi V.J. Chari, Bridget A. Martell, Jonathan L. Gross, Sherilyn B. Cook, Sudhir A. Shah, Walter A. Blättler, Sara J. McKenzie, and Victor S. Goldmacher: Immunoconjugates containing novel maytansinoids: promising anti-cancer drugs. Cancer Res. 52, 127-131 (1992).
John M. Lambert, Peter D. Senter, Annie Yau-Young, Walter A. Blättler, and Victor S. Goldmacher: Purified immunotoxins that are reactive with human lymphoid cells. J. Biol. Chem. 250, 12035-12041 (1985).

What is claimed is:

1. A method for identifying an agent that modulates the activity of a cancer-related gene comprising:
(a) contacting a compound with a cell expressing a gene that corresponds to a polynucleotide having a sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768 and under conditions promoting the expression of said gene; and
(b) determining a difference in expression of said gene relative to when said compound is not present
thereby identifying an agent that modulates the activity of a cancer-related gene.

2. The method of claim 1, wherein said gene comprises a sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768.

3. The method of claim 1, wherein the cell is a cancer cell.

4. The method of claim 1, wherein said change in expression is a change in amount of a mRNA produced.

5. The method of claim 1, wherein said change in expression is a change in amount of a polypeptide produced.

6. The method of claim 1, wherein said change in expression is a decrease in expression.

7. A method for determining the cancerous status of a cell, comprising determining an increase in the level of expression in said cell of a gene that corresponds to a polynucleotide having a sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768 wherein an elevated expression relative to a known non-cancerous cell indicates a cancerous state or potentially cancerous state.

8. An antibody specific for a polypeptide comprising an amino acid sequence selected from the group consisting of SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

9. An immunoconjugate comprising a cytotoxic agent and an antibody specific for a polypeptide comprising an amino acid sequence selected from SEQ ID NO: 82-109, 112, 114, 116, 119, 122, 124, 126, 128, 134-137, 139, 144, 145, 153-158, 166-170, 178-184, 189, 190, 192, 194, 205-210, 217-221, 231-234, 251-262, 270-272, 299-309, 320-325, 345-352, 360-364, 371-373, 430-448, 477-488, 505-509, 524-528, 560-572, 584-586, 610-615, 634-643, 656-664, 685-695, 698, 700, 702, 705, 707, 709, 711, 713, 715, 718, 722, 724, 727, 729, 731, 733, 735, 737, 739, 741, 743, 746, 749, 753, 755, 757, 759, 761, 763, 766 and 769.

10. The immunoconjugate of claim 9, wherein said cytotoxic agent is a member selected from the group consisting of a calicheamicin, a maytansinoid, an adozelesin, a cytotoxic protein, a taxol, a taxotere, a taxoid and DC1.

11. The immunoconjugate of claim 10, wherein said calicheamicin is calicheamicin γ₁^{I}, N-acetyl gamma calicheamicin dimethyl hydrazide or calicheamicin θ₁^{I}.

12. The immunoconjugate of claim 10, wherein said maytansinoid is DM1.

13. The immunoconjugate of claim 10, wherein said cytotoxic protein is ricin, abrin, gelonin, pseudomonas exotoxin or diphtheria toxin.

14. The immunoconjugate of claim 10, wherein said taxol is paclitaxel.

15. The immunoconjugate of claim 10, wherein said taxotere is docetaxel.

16. A method for inhibiting cancer cell proliferation comprising contacting a cancerous cell with an agent having activity against an expression product encoded by a gene sequence selected from the group consisting of SEQ ID NO: 1-81, 110, 111, 113, 115, 117, 118, 120, 121, 123, 125, 127, 129-133, 138, 140-143, 146-152, 159-165, 171-177, 185-188, 191-193, 195-204, 211-216, 222-230, 235-250, 263-269, 273-298, 310-319, 326-344, 353-359, 365-370, 374-429, 449-476, 489-504, 510-523, 529-559, 573-583, 587-609, 616-633, 644-655, 665-684, 696, 697, 699, 701, 703, 704, 706, 708, 710, 712, 714, 716, 717, 719, 720, 721, 723, 725, 726, 728, 730, 732, 734, 736, 738, 740, 742, 744, 745, 747, 748, 750, 751, 752, 754, 756, 758, 760, 762, 764, 765, 767 and 768.

17. The method of claim 16, wherein said expression product is a polypeptide.

18. The method of claim 16, wherein said agent is an antibody of claim 7.

19. The method of claim 16, wherein said agent is an immunoconjugate of claim 8.

20. The method of claim 16, wherein said contacting occurs in vivo.
